(19) United States
(12) Patent Application Publication
(10) Pub. No.: US 2018/0085051 A1
(43) Pub. Date: Mar. 29, 2018

(54) DEVICE CONTROL APPARATUS AND DEVICE CONTROL METHOD

(71) Applicant: YAMAHA CORPORATION, Hamamatsu-shi (JP)

(72) Inventors: Takahiro KAWASHIMA, Hamamatsu-shi (JP); Morito MORISHIMA, Fukuroi-shi (JP)

(21) Appl. No.: 15/713,998

(22) Filed: Sep. 25, 2017

(30) Foreign Application Priority Data
Sep. 28, 2016 (JP) 2016-189274
Sep. 30, 2016 (JP) 2016-192951

(51) Int. Cl. A61B 5/00; A61G 7/065; A61B 5/11; H04R 1/20

(52) U.S. Cl. CPC A61B 5/4815; A61B 5/4809; H04R 1/20; A61B 5/11; A61B 5/6891; A61G 7/065

(57) ABSTRACT

A device control apparatus includes: a receiving unit that receives an output signal of a pressure sensor installed in bedding; a determining unit that determines control information corresponding to the output signal from a plurality of sets of control information for device control; and a device control unit that controls a control target device using the control information corresponding to the output signal.

### BACKGROUND OF THE INVENTION

### Field of the Invention

The present invention relates to a technique for controlling a device.

Priority is claimed on Japanese Patent Application No. 2016-189274, filed Sep. 28, 2016, and Japanese Patent Application No. 2016-192951, filed Sep. 30, 2016, the content of which is incorporated herein by reference.

### Description of Related Art

Japanese Unexamined Patent Application, First Publication No. 2011-45637 (to be referred to as Patent Document 1 hereinafter) discloses a bed with a nurse call system that controls a nurse call slave unit in accordance with operations to the bed by the user. When a vibration sensor disposed in the bed detects a tap on the bed, this bed with a nurse call system transmits a call signal from the nurse call slave unit. Therefore, the user of this bed with a nurse call system can control the nurse call slave unit when in a lying-down state.

Japanese Unexamined Patent Application, First Publication No. 2003-87899 (to be referred to as Patent Document 2 hereinafter) discloses an acoustic processing device that outputs stereo sound in accordance with a sound signal from loudspeakers provided in a pillow.

When the user's head is held in a predetermined direction at a predetermined position on the pillow, this acoustic processing device performs processing on the sound signal so that the user feels that the stereo sound output from the loudspeakers in the pillow is coming from predetermined locations on the ceiling side.

The bed with a nurse call system disclosed in Patent Document 1 executes only the one control of transmitting a call signal from the nurse call slave unit in accordance with an operation to the user's bed.

Accordingly, when the user of this bed with a nurse call system performs a device control other than transmission of a call signal (for example, control of an audio device or control of an illumination device), it is necessary to rise up from the lying-down state, approach the control target device, and directly operate the device, or rise up from the lying-down state, pick up the remote control, and operate the remote control. For this reason, technology is desired that would allow the user to carry out a plurality of device controls while remaining in a lying-down state.

In the acoustic processing device disclosed in Patent Document 2, even if the orientation of the user's head changes, the processing applied to the sound signal does not change. For this reason, when the orientation of the user's head changes, it is difficult for this acoustic processing device to achieve the effect of having the user hear the sound output from the loudspeakers as stereo sound. That is, this acoustic processing device has the problem of no longer being able to impart a predetermined effect to a user when the orientation of the user's head changes.

### SUMMARY OF THE INVENTION

The present invention has been achieved in view of the aforementioned circumstances. An exemplary object of the present invention is to provide technology that can achieve a plurality of device controls while a user is in a lying-down state. Another exemplary object of the present invention is to provide technology in which even if the orientation of the user's head changes, a control target device imparts a predetermined effect to the user.

A device control apparatus according to one aspect of the present invention includes: a receiving unit that receives an output signal of a pressure sensor installed in bedding; a determining unit that determines control information corresponding to the output signal from a plurality of sets of control information for device control; and a device control unit that controls a control target device using the control information corresponding to the output signal.

A device control method according to one aspect of the present invention includes: receiving an output signal of a pressure sensor installed in bedding; determining control information corresponding to the output signal from a plurality of sets of control information for device control; and controlling a control target device using the control information corresponding to the output signal.

According to the above aspect, a user can change control information for device control by changing the pressure with respect to the bedding. For this reason, the user can execute a plurality of device control procedures in a lying-down state.

### BRIEF DESCRIPTION OF THE DRAWINGS

FIG. 1 is a diagram that shows the overall constitution of a device control system including a device control apparatus according to an embodiment A1 of the present invention.

FIG. 2 is a diagram that shows an example of a pressure sensor in the embodiment A1.

FIG. 3 is a diagram that shows a device control apparatus in the embodiment A1.

FIG. 4 is a table that shows an example of a control information table in the embodiment A1.

FIG. 5 is a flowchart for describing the operation of the device control apparatus in the embodiment A1.

FIG. 6 is a graph for describing the operation of a tap detecting unit in the embodiment A1.

FIG. 7 is a diagram that shows an example in which the pressure sensor includes four pressure sensors in the embodiment A1.

FIG. 8 is a diagram that shows the overall constitution of a device control system including a device control apparatus according to an embodiment B1 of the present invention.

FIG. 9 is a diagram that shows an example of pressure sensors in the embodiment B1.

FIG. 10 is a diagram that shows the state of a user facing right in the embodiment B1.

FIG. 11 is a diagram that shows the state of the user facing left in the embodiment B1.

FIG. 12 is a diagram that shows a loudspeaker unit viewed from a bed side in the embodiment B1.

FIG. 13 is a diagram that shows a device control apparatus in the embodiment B1.

FIG. 14 is a table that shows an example of a head orientation judgment table in the embodiment B1.

FIG. 15 is a graph that shows an example of judging the orientation of the head based on the head orientation judgment table in the embodiment B1.

FIG. 16 is a table that shows an example of a device control table in the embodiment B1.

FIG. 17 is a flowchart for describing the operation of the device control apparatus in the embodiment B1.

FIG. 18 is a table that shows an example of a device control table in the embodiment B1.

FIG. 19 is a diagram that shows a loudspeaker unit in the embodiment B1.

FIG. 20 is a table that shows an example of a device control table in the embodiment B1.

FIG. 21 is a diagram that shows a pillow with loudspeakers in the embodiment B1.

FIG. 22 is a table that shows an example of a device control table in the embodiment B1.

FIG. 23 is a diagram that shows an example of judging the orientation of the head using a pressure sensor in the embodiment B1.

FIG. 24 is a graph that shows an example of output signals when a leftward movement has occurred, in the embodiment B1.

FIG. 25 is a diagram that shows the overall constitution of a device control apparatus according to an embodiment C1 of the present invention.

### DETAILED DESCRIPTION OF THE INVENTION

Hereinbelow, embodiments for carrying out the invention will be described with reference to the drawings. The dimensions and scale of each component in the diagrams suitably differ from the actual dimensions and scale. Since the embodiments described below are preferred specific examples of the present invention, various preferred technical restrictions are imposed on the embodiments. However, the scope of the present invention is not limited to the embodiments unless specified in the following description.

### Embodiment A1

FIG. 1 is a diagram that shows the overall constitution of a device control system 11000 that includes a device control apparatus 1100 according to an embodiment A1 of the present invention. The device control system 11000 includes the device control apparatus 1100, a pressure sensor 1200, and an audio device 1500. The audio device 1500 includes an audio control unit 1501 and loudspeakers 1502 and 1503. The audio control unit 1501 outputs music such as a song from the loudspeakers 1502 and 1503.

The device control system 11000 supports remote operation of the audio device 1500 by a user 1E who is in a lying-down state on a bed 15. The pressure sensor 1200 is for example a sheet-shaped piezoelectric device. The pressure sensor 1200 is disposed at the bottom portion of the mattress of the bed 15, for example. The bed 15 is one example of bedding. The bedding is not limited to a bed and may be suitably changed. For example, the bedding may also be a futon.

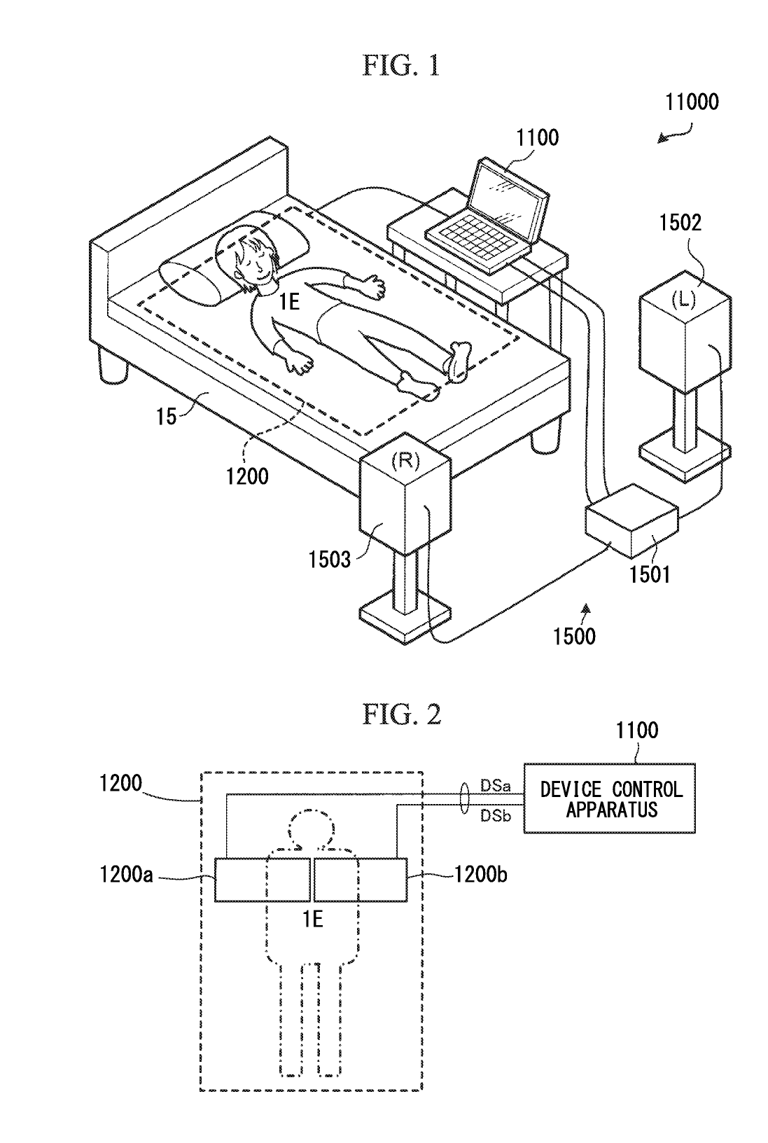

FIG. 2 is a diagram that shows an example of the pressure sensor 1200. In FIG. 2, the pressure sensor 1200 includes pressure sensors 1200a to 1200b.

The pressure sensors 1200a to 1200b are an example of a plurality of first pressure sensors disposed under the mattress of the bed 15 so as not to overlap each other.

The pressure sensor 1200a is disposed in a region where the right hand or right arm of the user 1E is positioned (to be referred to as the “right hand region” hereinafter) when the user 1E is in a facing-up (supine) state on the bed 15.

The pressure sensor 1200b is disposed in a region where the left hand or left arm of the user 1E is positioned (to be referred to as the “left hand region” hereinafter) when the user 1E is in a facing-up state on the bed 15.

When the user 1E is lying down, the pressure sensors 1200a to 1200b detect pressure changes that occur from the user 1E's heart rate, respiration, and physical movement, as biological information including respective components. In the embodiment A1, changes in a person's posture while in bed such as turning over are referred to as physical movement.

The pressure sensor 1200a outputs an output signal DSa on which the biological information is superimposed. When the user 1E lightly hits, in other words, taps, with a hand or foot the right hand region, the tap component indicating a pressure change corresponding to the tap to the right hand region is superimposed on the output signal DSa of the pressure sensor 1200a. Hereinbelow, a tap to the right hand region is referred to as a “right tap”.

The pressure sensor 1200b outputs an output signal DSb on which the biological information is superimposed. When the user 1E taps the left hand region, the tap component indicating a pressure change corresponding to the tap to the left hand region is superimposed on the output signal DSb of the pressure sensor 1200b. Hereinbelow, a tap to the left hand region is referred to as a “left tap”.

FIG. 1 and FIG. 2 for convenience show a constitution in which the output signals DSa and DSb are conveyed by wires to the device control apparatus 1100. However, one or both of the output signals DSa and DSb may also be conveyed wirelessly.

The device control apparatus 1100 controls the audio device 1500 on the basis of an output signal output from the pressure sensor 1200. Specifically, the device control apparatus 1100 determines the control information corresponding to the output signal of the pressure sensor 1200, from the plurality of sets of control information for device control. The device control apparatus 1100 controls the audio device 1500 using the control information corresponding to the output signal of the pressure sensor 1200. The device control apparatus 1100 is for example a portable terminal, a personal computer or a dedicated apparatus for device control.

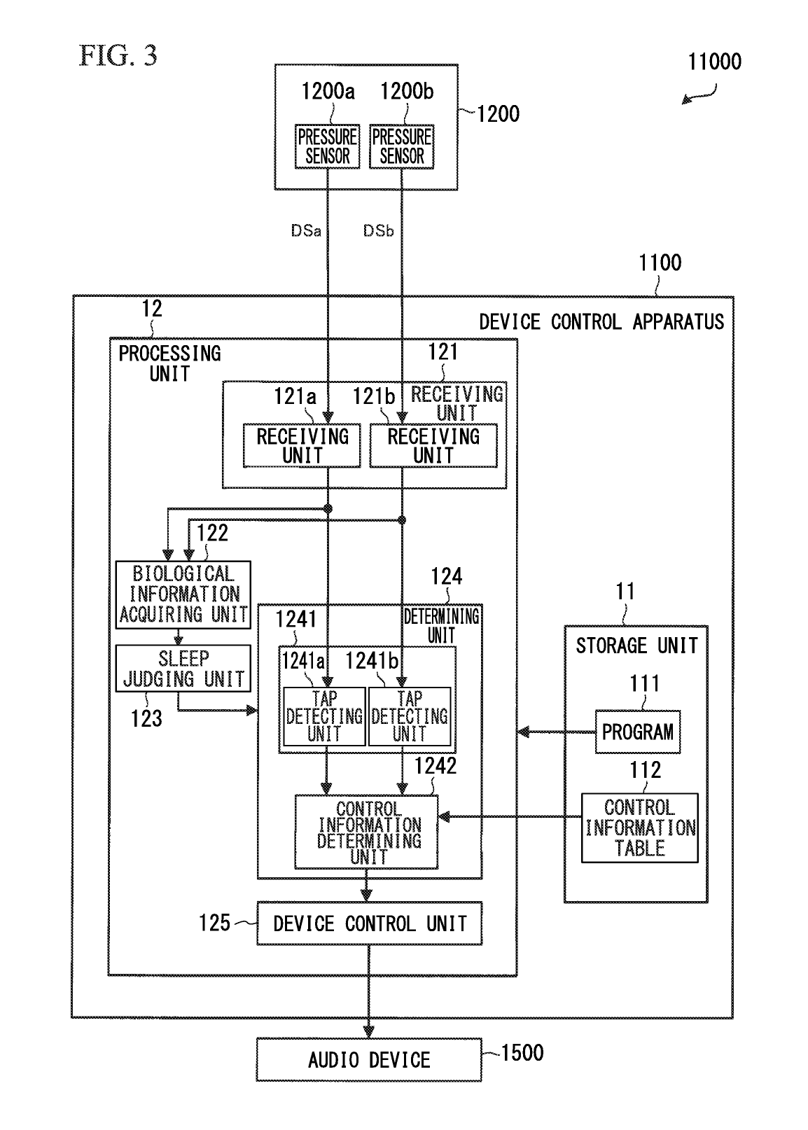

FIG. 3 is a diagram that chiefly shows the device control apparatus 1100 in the device control system 11000. The device control apparatus 1100 includes a storage unit 11 and a processing unit 12.

The storage unit 11 is an example of a computer-readable recording medium.

Moreover, the storage unit 11 is a non-transitory recording medium. The storage unit 11 is, for example, a recording medium of any publicly known form such as a semiconductor recording medium, a magnetic recording medium or an optical recording medium, or a recording medium in which these recording media are combined. In this specification, a “non-transitory” recording medium includes all computer-readable recording media except a recording medium such as a transmission line that temporarily stores a transitory, propagating signal, and does not exclude volatile recording media.

The storage unit 11 stores a program 111 and a control information table 112.

The program 111 defines the operation of the device control apparatus 1100. The program 111 may be provided in the form of distribution via a communication network (not shown) and subsequently installed in the storage unit 11.

The control information table 112 stores the correspondence relation between the plurality of sets of control information for device control and tap patterns.

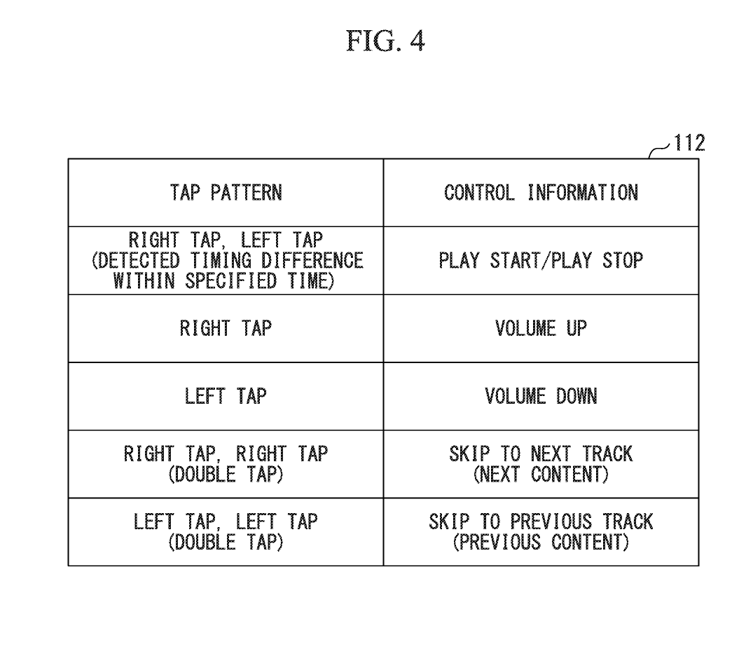

FIG. 4 is a table that shows an example of the control information table 112. In the control information table 112, the plurality of sets of control information for device control are associated with tap patterns, respectively. In the example of FIG. 4, the plurality of sets of control information for device control include play start/play stop, volume up, volume down, skip to next track (next content), and skip to previous track (previous content).

The processing unit 12 is a processing apparatus (computer) such as a central processing unit (CPU). The processing unit 12, by reading and executing the program 111 stored in the storage unit 11, realizes the receiving unit 121, a biological information acquiring unit 122, a sleep judging unit 123, the determining unit 124, and a device control unit 125.

The receiving unit 121 receives the output signal of the pressure sensor 1200 disposed on the bed 15. The receiving unit 121 includes receiving units 121a to 121b having a one-to-one correspondence with the pressure sensors 1200a to 1200b. The receiving unit 121a receives the output signal DSa of the pressure sensor 1200a. The receiving unit 121b receives the output signal DSb of the pressure sensor 1200b.

The biological information acquiring unit 122 acquires biological information including each component of heart rate, respiration, and physical movement from the output signal DSa and the output signal DSb. For example, the biological information acquiring unit 122 extracts a frequency component corresponding to the frequency range of a person's heart rate and the frequency component corresponding to the frequency range of a person's physical movement from each of the output signal DSa and the output signal DSb. The biological information acquiring unit 122 generates biological information including these frequency components. The biological information acquiring unit 122 may also acquire the biological information from either one of the output signal DSa and the output signal DSb.

The sleep judging unit 123 judges whether or not the user 1E has entered sleep on the basis of the biological information acquired by the biological information acquiring unit 122. For example, the sleep judging unit 123 first extracts the physical movement component of the user 1E from the biological information. Subsequently, the sleep judging unit 123 judges that the user 1E has entered sleep when a state in which the physical movement component is at or below a predetermined level has continued for a predetermined time.

The sleep judging unit 123 may also judge whether or not the user 1E has gone to sleep on the basis of the physical movement of the user 1E and the heart rate period of the user 1E. In the process of a person going to sleep, the heart rate period gradually becomes longer. Therefore, when the heart rate period has become longer than the heart rate period at the time of lying down by a predetermined time or more, and a state in which the physical movement component is at or below a predetermined level has continued for a predetermined time, the sleep judging unit 123 judges that the user 1E has gone to sleep. Also, since the respiratory period becomes longer during sleep as with the heart rate period, the respiratory period may be used instead of the heart rate period. Moreover, both periods may also be used.

The determining unit 124 determines the control information in accordance with the output signal received by the receiving unit 121, from among the plurality of sets of control information stored in the control information table 21. The determining unit 124 includes a tap detecting unit 1241 and a control information determining unit 1242.

The tap detecting unit 1241 detects a tap on the bed 15 on the basis of the output signal received by the receiving unit 121. The tap detecting unit 1241 includes tap detecting units 1241a to 1241b having a one-to-one correspondence with the pressure sensors 1200a to 1200b. The tap detecting unit 1241a detects a right tap on the basis of the output signal DSa. The tap detecting unit 1241b detects a left tap on the basis of the output signal DSb.

The control information determining unit 1242, on the basis of a tap detected by the tap detecting unit 1241, determines the control information corresponding to the output signal of the output sensor 1200 from the plurality of sets of control information in the control information table 112 (refer to FIG. 4). For example, the control information determining unit 1242, on the basis of the tap pattern, determines the control information corresponding to the output signal of the output sensor 1200 from the plurality of sets of control information in the control information table 112.

For this reason, by the user 1E changing the tap pattern to the bed 15, it is possible to change the control information corresponding to the output signal of the pressure sensor 1200.

When the sleep judging unit 123 has determined that the user 1E has gone to sleep, the determining unit 124 suspends the determination of control information corresponding to the output signal of the pressure sensor 1200. For this reason, it is possible to render ineffective taps to the bed 15 performed unconsciously by the user 1E after having gone to sleep.

The device control unit 125 controls the audio device 1500 using the control information corresponding to the output signal of the pressure sensor 1200. The audio device 1500 is an example of a control target device (device to be controlled). The audio device 1500 outputs music that encourages the user 1E to go to sleep. The audio output by the audio device 1500 is not limited to music and can be suitably changed.

Next, the operation will be described.

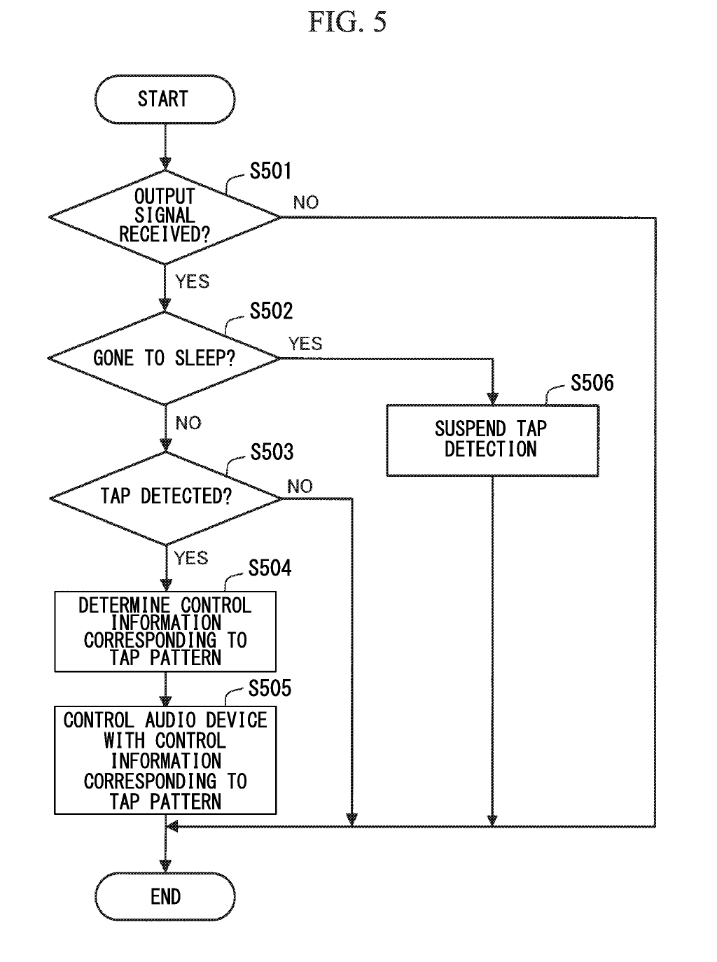

FIG. 5 is a flowchart for describing the operation of the device control apparatus 1100. The device control apparatus 1100 repeats the operation shown in FIG. 5.

If the user 1E lies down on the bed 15, the pressure sensor 1200a will output an output signal DSa, and the pressure sensor 1200b will output an output signal DSb.

When the receiving unit 121a receives the output signal DSa, and the receiving unit 121b receives the output signal DSb (Step S501: YES), the output signal DSa is supplied from the receiving unit 121a to the biological information acquiring unit 122 and the tap detecting unit 1241a, and the output signal DSb is supplied from the receiving unit 121b to the biological information acquiring unit 122 and tap detecting unit 1241b.

The biological information acquiring unit 122 acquires biological information including respective components of the heart rate and physical movement from the output signal DSa and the output signal DSb. The sleep judging unit 123 determines whether or not the user 1E has gone to sleep on the basis of the biological information acquired by the biological information acquiring unit 122.

When the sleep judging unit 123 has judged that the user 1E is not asleep (Step S502: NO), the sleep judging unit 123 supplies wakefulness information indicating that the user 1E is in a wakeful state to the determining unit 124.

When the determining unit 124 receives the wakefulness information, the tap detecting unit 1241a executes an operation for detecting a right tap on the basis of the output signal DSa, and the tap detecting unit 1241b executes an operation for detecting a left tap on the basis of the output signal DSb.

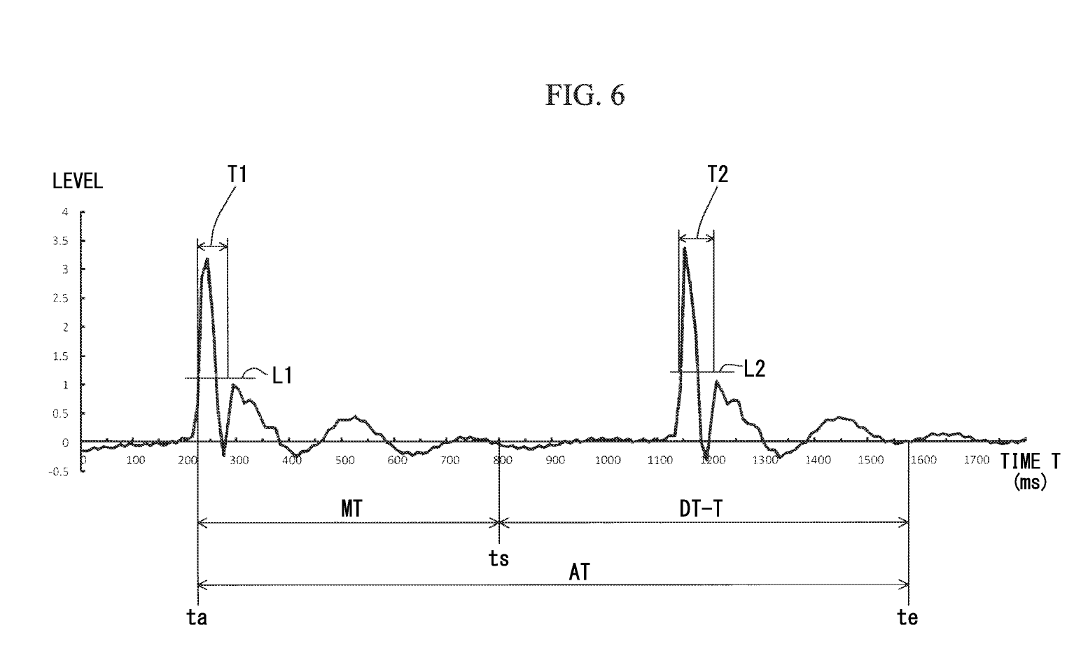

FIG. 6 is a graph for describing the operation of the tap detecting unit 1241a.

FIG. 6 shows the operation for detecting a right-hand tap. The right-hand tap is detected, when a right-hand tap has been performed, in the case where the time (to be referred to as “first continuous time” hereinafter) during which the level of the output signal DSa (voltage level) continuously exceeds a first threshold value L1 is around 40 ms.

Moreover, FIG. 6 shows the operation for detecting a second right-hand tap. The second right-hand tap is detected, when a right-hand double tap has been performed, in the case where the time (hereinbelow referred to as “second continuous time”) during which the level of the output signal DSa corresponding to the second right-hand tap continuously exceeds a second threshold L2 is around 40 ms.

Also, in order to determine whether the change in the level of the output signal DSa is due to a right-hand tap or due to the user turning over in bed, a first time T1 and a second time T2 are used. As an example of the first time T1 and the second time T2, 100 ms is used. Note that the first time T1 and the second time T2 are not limited to 100 ms, and need only be longer than 40 ms.

When the first continuous time is less than the first time T1, the tap detecting unit 1241a judges that a right tap has been performed, and detects the right tap. On the other hand, if the first continuous time is equal to or longer than the first time T1, the tap detecting unit 1241a judges that the user 1E has turned over.

Moreover, the tap detecting unit 1241a uses as a double tap detection period DT-T the period between point in time ts and point in time te. The point in time ts is the time at which time MT has elapsed from point in time ta at which the level of the output signal DSa exceeded the first threshold value L1. The point in time te is the time at which time AT (AT>MT) has elapsed from the point in time ta.

When the second continuous time is less than the second time T2 during the double tap detection period DT-T, the tap detecting unit 1241a judges that the second right tap of the double tap has been performed, and detects the second right tap of the double tap. On the other hand, if the second continuous time is equal to or greater than the second time T2, the tap detecting unit 1241a judges that the user 1E has turned over in bed.

The tap detecting unit 1241a, upon detecting a right tap, outputs a right-tap detection result to the control information determining unit 1242.

The first threshold value L1 and the second threshold value L2 may be a common value or may be different values. The first time T1 and the second time T2 may be a common value or may be different values.

A description of the operation of the tap detecting unit 1241b is carried out by replacing “right tap” in the operation description of the tap detecting unit 1241a with “left tap”.

When a tap is detected by the tap detecting unit 1241 in Step S503 (Step S503: YES), the control information determining unit 1242, from the plurality of sets of control information in the control information table 112, determines the control information corresponding to the tap pattern detected by the tap detecting unit 1241 to be the control information corresponding to the output signal of the pressure sensor 1200 (Step S504).

For example, when a right tap and a left tap are detected, and the difference between the timing at which the control information determining unit 1242 has received the right tap detection result and the timing at which the control information determining unit 1242 has received the left tap detection result is within a specified time, the control information determining unit 1242 determines the control information indicating “play start/play stop” to be the control information corresponding to the output signal of the pressure sensor 1200.

When a right tap is detected, the control information determining unit 1242 determines the control information indicating “volume up” to be the control information corresponding to the output signal of the pressure sensor 1200.

When a left tap is detected, the control information determining unit 1242 determines the control information indicating “volume down” to be the control information corresponding to the output signal of the pressure sensor 1200.

When a right double tap (right tap, right tap) is detected, the control information determining unit 1242 determines the control information indicating “skip to next track (next content)” to be the control information corresponding to the output signal of the pressure sensor 1200.

When a left double tap (left tap, left tap) is detected, the control information determining unit 1242 determines the control information indicating “skip to previous track (previous content)” to be the control information corresponding to the output signal of the pressure sensor 1200.

The control information determining unit 1242 outputs the control information corresponding to the output signal of the pressure sensor 1200 to the device control unit 125.

The device control unit 125 controls the audio device 1500 using the control information corresponding to the output signal of the pressure sensor 1200 (Step S505).

For example, the device control unit 125, upon receiving the control information indicating “play start/play stop”, outputs control information indicating “play start/play stop” to the audio device 1500. The device control unit 125 outputs the control information by wires or wirelessly to the audio device 1500.

In the audio device 1500, the audio control unit 1501, upon receiving control information indicating “play start/play stop”, starts playback of music in the case of music playback not being performed, and stops music playback in the case of music playback being performed. Music is one example of content.

Also, the audio control unit 1501, upon receiving control information indicating “volume up”, increases the volume of the music by one step.

The audio control unit 1501, upon receiving control information indicating “volume down”, decreases the volume of the music by one step.

The audio control unit 1501, upon receiving control information indicating “next track (next content)”, changes (skips) the track to be played from the track currently being played to the next track.

The audio control unit 1501, upon receiving control information indicating “previous track (previous content)”, changes (skips) the track to be played from the track currently being played to the previous track.

On the other hand, when the sleep judging unit 123 has judged that the user 1E has gone to sleep in Step S502 (Step S502: YES), the sleep judging unit 123 supplies sleep onset information indicating that the user 1E has entered the state of sleep to the determining unit 124.

When the determining unit 124 receives the sleep onset information, the tap detecting units 1241a and 1241b suspends tap detection (Step S506). For this reason, the operation of determining control information corresponding to the output signal of the pressure sensor 1200 stops. Thereby, it is possible to render ineffective taps to the bed 15 performed unconsciously by the user 1E after going to sleep.

When it is determined in Step S501 that the receiving unit 121 has not received the output signal DSa and the output signal DSb (Step S501: NO), and when it is determined in Step S503 that the tap detecting unit 1241 has not detected a tap, the operation shown in FIG. 5 ends.

According to the embodiment A1, the determining unit 124 determines the control information corresponding to the output signal of the pressure sensor 1200 received by the receiving unit 121 from the plurality of sets of control information stored in the control information table 112.

For this reason, it is possible for the user 1E to switch the control information for device control by for example changing the manner of applying pressure to the bed. Thereby, the user 1E can execute a plurality of device operations in a state of lying down.

Accordingly, when the user 1E is a healthy person, compared to the case of the healthy person getting up from the bed 15 to perform device control, it is possible to prevent interference with a healthy person going to sleep.

In contrast, when the user 1E is a person who requires assistance, the person who requires assistance can perform a plurality of device operations without getting up from the bed 15.

The control information determining unit 1242 determines the control information corresponding to the output signal of the pressure sensor 1200 from the plurality of sets of control information on the basis of the taps detected by the tap detecting unit 1241.

For this reason, it is possible for the user 1E to switch the control information for device control by changing the tapping on the bed 15. Thereby, the user 1E can execute a plurality of device operations in a state of lying down.

The control information determining unit 1242 determines the control information corresponding to the output signal of the pressure sensor 1200 from the plurality of sets of control information on the basis of the tap pattern.

For this reason, the user 1E can switch the control information for device control by changing the pattern of tapping on the bed 15. Thereby, the user 1E can execute a plurality of device operations in a state of lying down.

The present sensor 1200 includes the pressure sensor 1200a and the pressure sensor 1200b, which are disposed under the bed 15 so as not to overlap each other. The output signal of the pressure sensor 1200 includes the output signal DSa of the pressure sensor 1200a and the output signal DSb of the pressure sensor 1200b.

For this reason, the user 1E can change the control information for controlling a control target device by suitably changing the respective pressure state on different locations of the bed 15 while in a lying-down state.

The biological information acquiring unit 122 acquires biological information of the user 1E on the basis of the output signal of pressure sensor 1200.

For this reason, it becomes possible to acquire biological information of the user 1E from the output signal of the pressure sensor 1200 used in order to determine control information. Therefore, compared with the case of acquiring biological information of the user 1E on the basis of a signal different from the output signal of the pressure sensor 1200, it is possible to reduce the number of the signals that the device control apparatus 1100 receives.

The pressure sensor 1200 that detects tapping by the user 1E in order to control a device can also be made to serve as a sensor that detects biological information. For this reason, it becomes possible to achieve simplification of the constitution.

When the sleep judging unit 123 judges that user 1E has gone to sleep, the determining unit 124 suspends determination of control information corresponding to the output signal of pressure sensor 1200.

For this reason, it is possible to render ineffective operations to the bed 15 performed unconsciously by the user 1E after having gone to sleep.

### Modification Examples

The embodiment exemplified above may be modified in various respects. Specific modification examples will be exemplified below. Two or more examples which are arbitrarily selected from modification examples which are exemplified below may be appropriately combined as far as the examples do not conflict with each other.

### Modification Example A1

In Step S501, when the receiving unit 121 has received either one of the output signal DSa and the output signal DSb, the processing may proceed to Step S502.

### Modification Example A2

The control target device is not limited to an audio device and may be appropriately changed. For example, the control target device may be an air conditioner, an electric fan, a lighting device, an elevating bed, or nursing equipment.

### Modification Example A3

The plurality of sets of control information stored in the control information table 112 are not restricted to a plurality of sets of control information for one control target device.

For example, the control information table 112 may store first control information for controlling the audio device 1500, and second control information for controlling a lighting device. For example, second control information indicates “turn on light/turn off light”. In this case, the tap pattern corresponding to the first control information and the tap pattern corresponding to the second control information mutually differ. The lighting device, upon receiving the second control information that indicates “turn on light/turn off light”, will turn on the lighting if the lighting is off, and turn off the lighting if the lighting is on.

In addition, the control information table 112 may store, for each of a plurality of devices to be controlled, at least one piece of control information in association with a tap pattern.

According to the modification example A3, the user 1E becomes able to control a plurality of devices in a lying-down state.

### Modification Example A4

The plurality of sets (pieces) of control information for device control are not limited to the control information shown in FIG. 4 and may be appropriately changed. The number of sets of control information for device control is not limited to the number shown in FIG. 4 and may be appropriately changed. Also, the correspondence relation between control information and tap pattern is not limited to the correspondence relation shown in FIG. 4 and may be appropriately changed.

### Modification Example A5

The number of the pressure sensors that the pressure sensor 1200 includes is not limited to two and may be one or more. The greater the number of pressure sensors included in the pressure sensor 1200, the more combinations of tap patterns that become possible.

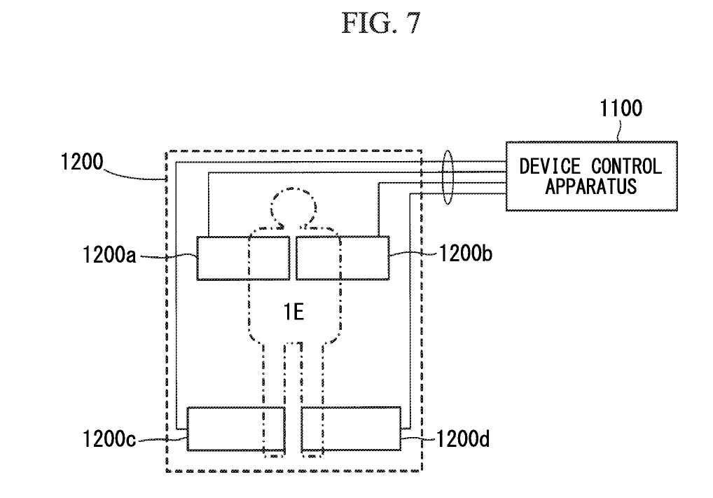

FIG. 7 is a diagram that shows an example in which the pressure sensor 1200 includes four pressure sensors, namely pressure sensors 1200a, 1200b, 1200c, and 1200d.

The pressure sensor 1200c is arranged in a region where the right foot of the user 1E is positioned (to be referred to as the “right foot region” hereinafter) when the user 1E is in a facing-up state on the bed 15. The pressure sensor 1200d is arranged in a region where the left foot of the user 1E is positioned (to be referred to as the “left foot region” hereinafter) when the user 1E is in a facing-up state on the bed 15. In this case, the pressure sensor 1200 can detect taps which the user 1E performs at each of the four regions, namely right hand region, a left hand region, right leg region, and left leg region. Therefore, it becomes possible to set control information for device control in accordance with a pattern of combination of taps at the four regions.

### Modification Example A6

One or both of the tap detecting units 1241a and 1241b may perform tap detection using a tap detection model generated by machine learning.

For example, the tap detecting unit 1241a generates a tap detection model by performing machine learning, using as learning data each of the output signal DSa when a right tap single tap is performed, and the output signal DSa when a right tap double tap is performed. A tap detection model is a model that indicates the relation between the output signals DSa, a right tap single tap, and a right tap double tap.

Using the tap detection model, the tap detecting unit 1241a determines a right tap single tap corresponding to the output signal DSa of the pressure sensor 1200R, and a right tap double tap corresponding to the output signal DSa of pressure sensor 1200R.

The tap detecting unit 1241b, when performing tap detection using a tap detection model generated by machine learning, executes an operation conforming to the operation of the tap detecting unit 1241a described above.

### Modification Example A7

The biological information acquiring unit 122 and sleep judging unit 123 may be omitted. In this case, Step S502 and Step S506 of FIG. 5 are skipped, and when the receiving unit 121a receives the output signal DSa, and the receiving unit 121b receives the output signal DSb in Step S501, the tap detecting unit 1241a executes an operation for detecting a right tap based on output signal DSa, and the tap detecting unit 1241b executes an operation for detecting a left tap based on the output signal DSb.

### Modification Example A8

All or some of the receiving unit 121, the biological information acquiring unit 122, the sleep judging unit 123, the determining unit 124, and the device control unit 125 may be realized by dedicated electronic circuits.

The following aspects are ascertained from at least one of the aforementioned embodiment A1 and the modifications A1 to A8.

A device control apparatus according to one aspect of the present invention includes: a receiving unit that receives an output signal of a pressure sensor installed in bedding; a determining unit that determines control information corresponding to the output signal from a plurality of sets of control information for device control; and a device control unit that controls a control target device using the control information corresponding to the output signal.

According to the above device control apparatus, a user can change control information for device control by changing pressure applied to the bedding. For this reason, the user can execute a plurality of device control procedures in a lying-down state.

In the above device control apparatus, the determining unit may include: a tap detecting unit that detects a tap on the bedding based on the output signal; and a control information determining unit that determines the control information corresponding to the output signal from the plurality of sets of control information, based on the tap.

According to the above device control apparatus, the user can change the control information for device control by changing the tap on the bedding. For this reason, the user can execute a plurality of device control procedures in a lying-down state.

In the above device control apparatus, the control information determining unit may determine the control information corresponding to the output signal from the plurality of sets of control information based on a pattern of the tap.

According to the above device control unit, the user can change the control information for device control by changing the pattern of the tap on the bedding. For this reason, the user can execute a plurality of device controls in a lying-down state.

In the above device control apparatus, the pressure sensor may include a plurality of first pressure sensors disposed under the bedding so as not to overlap each other, and the output signal may include a first output signal of each of the plurality of first pressure sensors.

According to the above device control apparatus, it is possible to change control information for controlling a control target device in accordance with the state of pressure at different locations of the bedding.

The above device control apparatus may further include: an acquiring unit that acquires biological information of a user of the bedding based on the output signal.

According to the above device control apparatus, it is possible to acquire biological information of the user from the output signal used for determining the control information. Therefore, it becomes possible to efficiently use the output signal compared to the case of acquiring biological information of the user on the basis of a signal different from the output signal.

The above device control apparatus may further include: a sleep judging unit that judges whether the user has gone to sleep based on the biological information. The determining unit may suspend determination of the control information corresponding to the output signal in a case where the sleep judging unit judges that the user has gone to sleep.

According to the above device control apparatus, it becomes possible to render ineffective operations to the bedding that are performed unconsciously by the user after having gone to sleep.

A device control method according to one aspect of the present invention includes: receiving an output signal of a pressure sensor installed in bedding; determining control information corresponding to the output signal from a plurality of sets of control information for device control; and controlling a control target device using the control information corresponding to the output signal.

According to the above device control method, a user can change control information for device control by changing pressure applied to the bedding. For this reason, the user can execute a plurality of device controls in a lying-down state.

### Embodiment B1

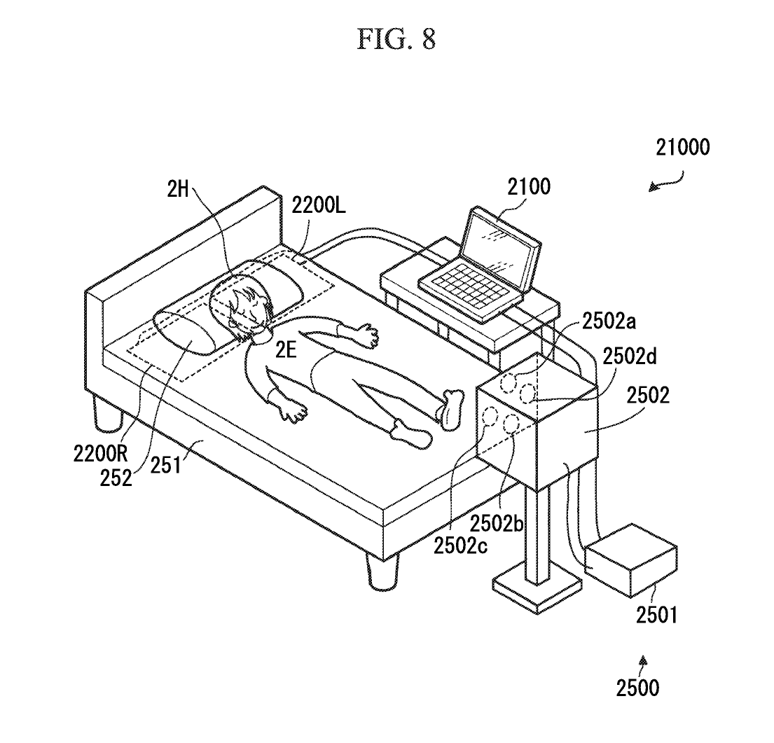

FIG. 8 is a diagram that shows the entire constitution of a device control system 21000 including a device control apparatus 2100 according to an embodiment B1 of the present invention. The device control system 21000 includes a device control apparatus 2100, pressure sensors 2200R and 2200L, and an audio device 2500.

The pressure sensors 2200R and 2200L are for example sheet-shaped piezoelectric devices. The pressure sensors 2200R and 2200L are disposed under a pillow 252 disposed on a bed 251. The pillow 252 is an example of bedding. The bedding is not limited to a pillow and may be suitably changed. For example, the bedding may be the bed 251 or a futon mat. When the bed 251 is used as the bedding, the pressure sensors 2200R and 2200L are disposed under the mattress portion opposite the pillow 252 on the bed 251. When a futon mat is used as the bedding, the pressure sensors 2200R and 2200L are disposed under the mattress portion opposite the pillow 252 on the futon mat.

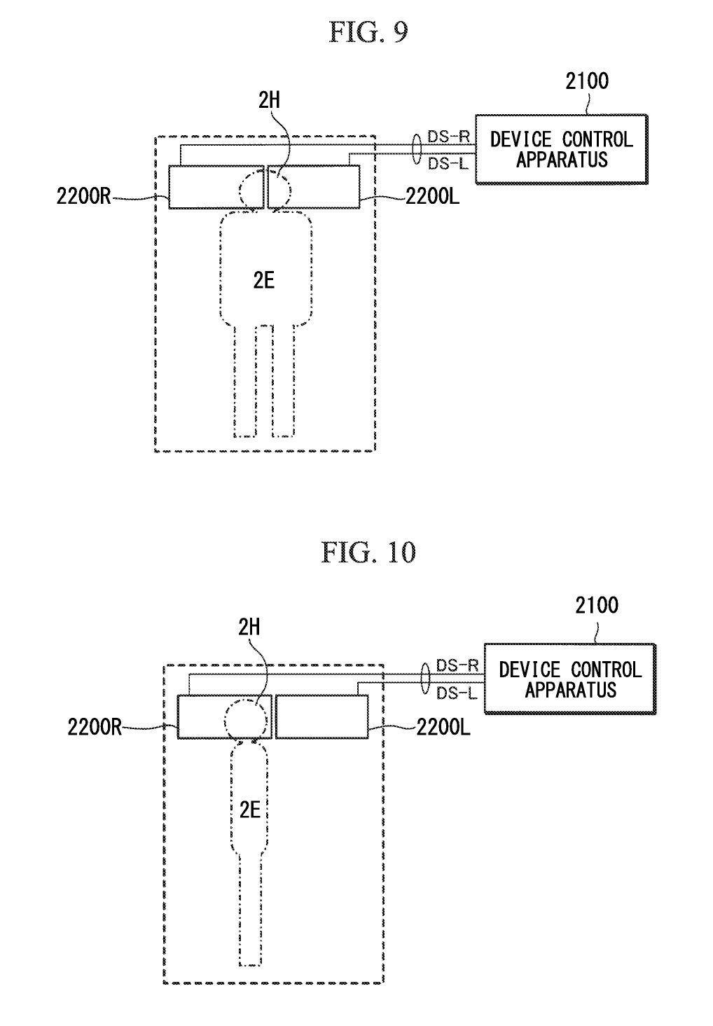

FIG. 9 is a diagram that shows an example of the pressure sensors 2200R and 2200L.

In the state where a user 2E is in a facing-up state on the bed 251 with the head 2H placed on the center of the pillow 252, the pressure sensor 2200R is disposed in a region on the right side of the user 2E from the center of the pillow 252 (to be referred to as the “right side region” hereinafter).

In the state where the user 2E is in the facing-up state, the pressure sensor 2200L is disposed in a region on the left side of the user 2E from the center of the pillow 252 (to be referred to as the “left side region” hereinafter).

When the user 2E is facing up (supine), as shown in FIG. 9, both pressure sensors 2200R and 2200L receive pressure from the head 2H of the user 2E. Furthermore in this case, the pressure sensors 2200R and 2200L detect pressure changes that occur from the user 2E's heart rate, respiration, and physical movement, as biological information including respective components. In the embodiment B1, changes in a person's posture while in bed such as turning over are referred to as physical movement.

For this reason, each of the output signal DS-R of the pressure sensor 2200R and the output signal DS-L of the pressure sensor 2200L includes a component resulting from pressure received from the head 2H and a component resulting from biological information (biological information of the user 2E).

As shown in FIG. 10, when the user 2E rotates from a facing-up state to the user 2E's right to change to a state of facing right, the pressure sensor 2200R receives the pressure from the head 2H, and the pressure sensor 2200L no longer receives pressure from the head 2H.

Therefore, the output signal DS-R of the pressure sensor 2200R includes the component resulting from the pressure received from the head 2H and the component resulting from biological information. In contrast, the output signal DS-L of the pressure sensor 2200L no longer includes either the component resulting from the pressure received from head 2H or the component resulting from biological information.

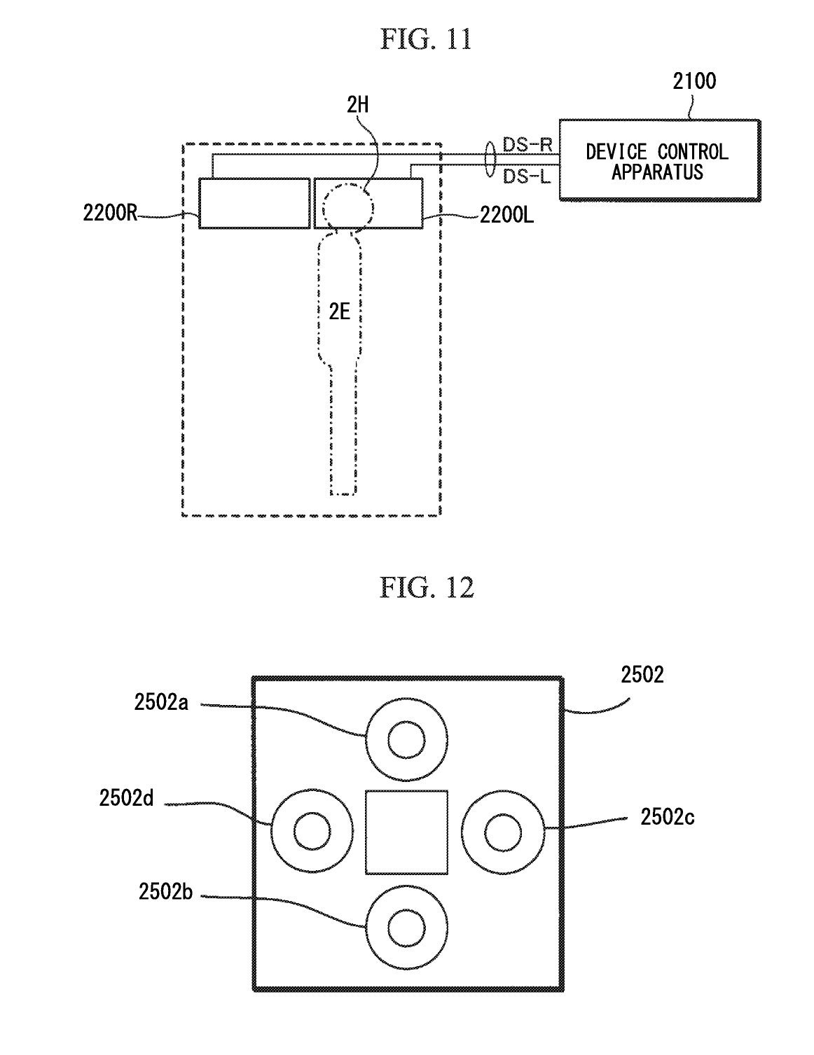

As shown in FIG. 11, when the user 2E rotates from a facing-up state to the user 2E's left to change to a state of facing left, the pressure sensor 2200L receives the pressure from the head 2H, and the pressure sensor 2200R no longer receives pressure from the head 2H.

Therefore, the output signal DS-L of the pressure sensor 2200L includes the component resulting from the pressure received from the head 2H and the component resulting from biological information. In contrast, the output signal DS-R of the pressure sensor 2200R no longer includes either the component resulting from the pressure received from head 2H or the component resulting from biological information.

Returning to FIG. 8, the audio device 2500 is an example of a control target device and a sound output apparatus. The audio device 2500 includes an audio control unit 2501 and a loudspeaker unit 2502.

The audio control unit 2501 outputs sound such as music from the loudspeaker unit 2502. The loudspeaker unit 2502 has loudspeakers 2502a to 2502d. The loudspeakers 2502a to 2502d are disposed so as to emit sound toward the bed 251.

FIG. 12 is a diagram that shows the loudspeaker unit 2502 viewed from the bed 251 side. As shown in FIG. 8 and FIG. 12, the loudspeaker 2502a is disposed at a position shifted vertically upward from the loudspeaker 2502b. The loudspeaker 2502c and the loudspeaker 2502d are aligned in a direction perpendicular to the vertical direction (hereinbelow referred to as the “horizontal direction”). The loudspeaker 2502c is disposed more to the right-hand side of the user 2E than the loudspeaker 2502d in the state where the user 2E is in a facing-up state.

Returning again to FIG. 8, the device control apparatus 2100 is for example a mobile terminal, a personal computer or a dedicated apparatus for device control. The device control apparatus 2100 judges the orientation of the head 2H of the user 2E on the basis of the output signal DS-R of the pressure sensor 2200R and the output signal DS-L of the pressure sensor 2200L. The device control apparatus 2100 controls the sound image of stereo sound output from the loudspeaker unit 2502 in accordance with the orientation of the head 2H of the user 2E.

FIG. 8 to FIG. 11 show the constitution in which the output signals DS-R and DS-L are conveyed by wires to the device control apparatus 2100. However, one or both of the output signals DS-R and DS-L may also be conveyed wirelessly.

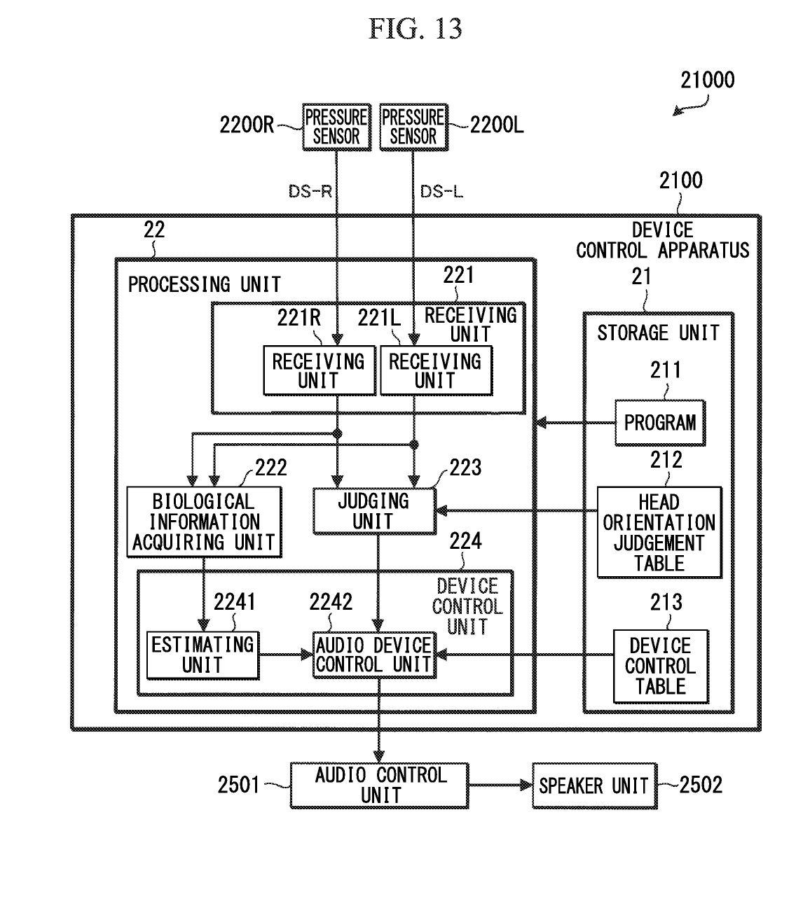

FIG. 13 is a diagram that chiefly shows the device control apparatus 2100 in the device control system 21000. The device control apparatus 2100 includes a storage unit 21 and a processing unit 22.

The storage unit 21 is an example of a computer-readable recording medium. Moreover, the storage unit 21 is a non-transitory recording medium. The storage unit 21 is, for example, a recording medium of any publicly known form such as a semiconductor recording medium, a magnetic recording medium or an optical recording medium, or a recording medium in which these recording media are combined. In this specification, a “non-transitory” recording medium includes all computer-readable recording media except a recording medium such as a transmission line that temporarily stores a transitory, propagating signal, and does not exclude volatile recording media.

The storage unit 21 stores a program 211, a head orientation judgment table 212, and a device control table 213.

The program 211 defines the operation of the device control apparatus 2100. The program 211 may be provided in the form of distribution via a communication network (not shown) and subsequently installed in the storage unit 21.

The head orientation judgment table 212 stores the relation of the output signal DS-R and the output signal DS-L, and the head orientation in association with each other.

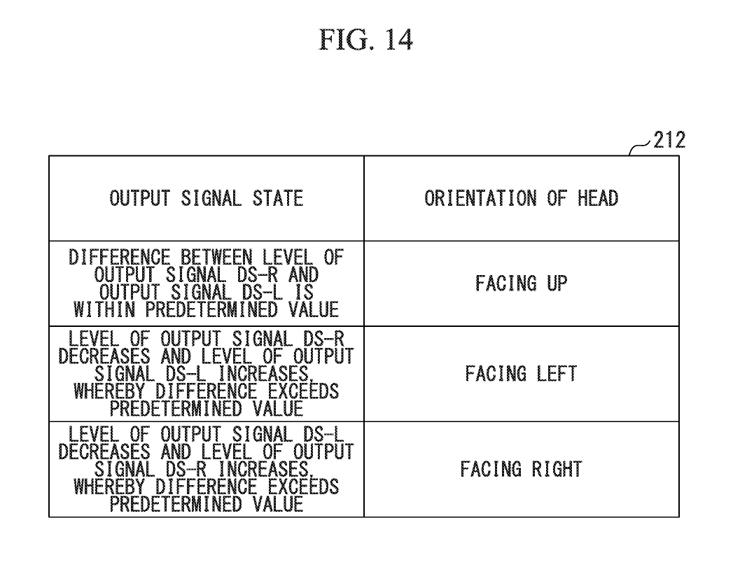

FIG. 14 is a table that shows an example of the head orientation judgment table 212. In the head orientation judgment table 212 shown in FIG. 14, facing up, facing left and facing right are used as the head orientations. FIG. 15 is a graph that shows a judgment example of head orientation based on the head orientation judgment table 212, specifically showing the judgment examples of the head facing up and facing left.

The device control table 213 stores the head orientation and setting information in association with each other.

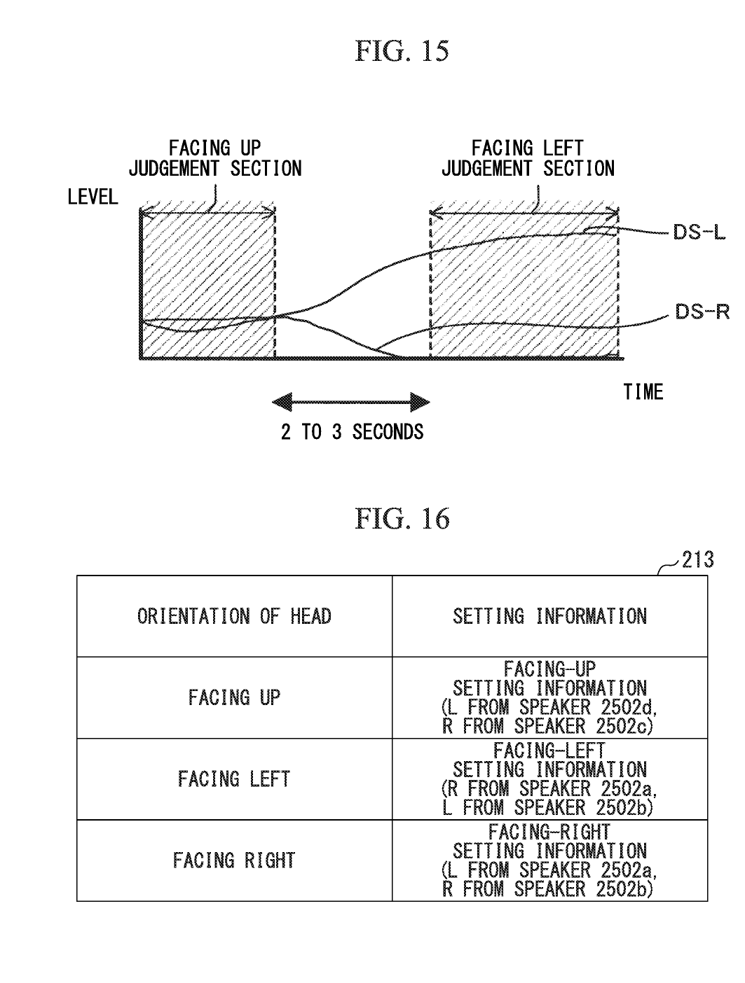

FIG. 16 is a table that shows an example of the device control table 213. In the device control table 213 shown in FIG. 16, setting information is shown for each head orientation. In the example shown in FIG. 16, the setting information is information indicating the loudspeaker to output the right (R) channel of stereo sound and the loudspeaker to output the left (L) channel of stereo sound. Hereinbelow, the right channel of stereo sound (that is, right (R) stereo sound) is referred to as the “R sound” and the left channel of stereo sound (that is, left (L) stereo sound) is referred to as the “L sound”.

The processing unit 22 is a processing apparatus (computer) such as a central processing unit (CPU). The processing unit 22, by reading and executing the program 211 stored in the storage unit 21, realizes the receiving unit 221, a biological information acquiring unit 222, a judging unit 223, and a device control unit 224.

The receiving unit 221 receives the output signal DS-R of the pressure sensor 2200R and the output signal DS-L of the pressure sensor 2200L. The receiving unit 221 includes a receiving unit 221R that corresponds to the pressure sensor 2200R and a receiving unit 221L that corresponds to the pressure sensor 2200L.

The receiving unit 221R receives the output signal DS-R of the pressure sensor 2200R. The output signal DS-R is output from the receiving unit 221R to the biological information acquiring unit 222 and the judging unit 223.

The receiving unit 221L receives the output signal DS-L of the pressure sensor 2200L. The output signal DS-L is output from the receiving unit 221L to the biological information acquiring unit 222 and the judging unit 223.

The biological information acquiring unit 222 acquires biological information including each of the components of heart rate and physical movement from the output signal DS-R and the output signal DS-L. For example, the biological information acquiring unit 222 extracts a frequency component corresponding to the frequency range of a person's heart rate and the frequency component corresponding to the frequency range of a person's physical movement from each of the output signal DS-R and the output signal DS-L. The biological information acquiring unit 222 generates biological information including these frequency components. The biological information acquiring unit 222 may also acquire biological information from either one of the output signal DS-R and the output signal DS-L. In this case, either one of the output signal DS-R and the output signal DS-L may be supplied to the biological information acquiring unit 222.

The judging unit 223 judges the orientation of the head 2H of the user 2E (hereinbelow simply referred to as “head 2H orientation”) on the basis of the output signal DS-R and the output signal DS-L. In the embodiment B1, the judging unit 223 judges the head 2H orientation referring to the head orientation judgment table 212.

The device control unit 224 controls the audio control unit 2501 in accordance with the head 2H orientation and the biological information. The device control unit 224 includes an estimating unit 2241 and an audio device control unit 2242.

The estimating unit 2241 estimates the stage of sleep of the user 2E from among three stages.

Generally, when going from a resting state to deep sleep, a person's heart rate period tends to become longer and fluctuations in the heart rate period tend to become smaller. In addition, when sleep becomes deep, physical movement will also decrease. Therefore, the estimating unit 2241 estimates the stage of sleep of the user 2E divided into a first stage, a second stage, and a third stage on the basis of the change in the heart rate period and the number of times of physical movement per unit of time which are based on the biological information obtained by the biological information acquiring unit 222. Here, sleep becomes deeper in the order of first stage, second stage, and third stage.

When the biological information acquiring unit 222 also acquires the respiration component as biological information, the estimating unit 2241 may also estimate the stage of sleep that the user 2E is in among a first stage, a second stage, or a third stage on the basis of the change in the respiration period, the change in the heart rate period, and the number of times of physical movement per unit of time. Relatedly, when going from a resting state to deep sleep, a person's respiration period tends to become longer and fluctuations in the respiration period tend to be smaller.

β waves are the most common type of brainwave when people are in an active state. a waves begin to appear when people relax. The frequency range of a waves is 8 Hz to 14 Hz. For example, when a person lies down and closes his eyes, a waves begin to appear. As a person further relaxes, the α waves gradually become larger. The stage from a person relaxing to α waves beginning to become larger corresponds to the first stage. That is, the first stage is the stage prior to the α waves becoming dominant.

Moreover, when a person's state heads toward sleep, the proportion of α waves in the person's brainwaves increases. However, before long the α waves diminish and θ waves, which are said to emerge when a person is in a meditation state or a drowsy state, begin to appear. The stage until this point corresponds to the second stage. That is, the second stage is the stage prior to θ waves becoming dominant. The frequency range of θ waves is 4 Hz to 8 Hz.

Subsequently, θ waves become dominant, and a person's state is almost that of sleep. When sleep further advances, δ waves, which are said to emerge when a person has entered deep sleep, begin to appear. The stage until this point corresponds to the third stage. That is, the third stage is the stage prior to δ waves becoming dominant. The frequency range of δ waves is 0.5 Hz to 4 Hz.

The audio device control unit 2242 controls the audio control unit 2501 in accordance with the head 2H orientation, and the stage of sleep of the user 2E.

The audio device control unit 2242 controls the loudspeaker that outputs the L sound (that is, the sound of the left (L) channel of stereo sound) and the loudspeaker that outputs the R sound (that is, the sound of the right (R) channel of stereo sound) according to the head 2H orientation, with reference to the device appliance control table 213 (refer to FIG. 16).

Also, the audio device control unit 2242 controls the volume of the sound output by the audio device 2500 in accordance with the stage of sleep of the user 2E. For example, the audio device control unit 2242 reduces the volume as the stage of sleep becomes deeper.

Next, the operation will be described.

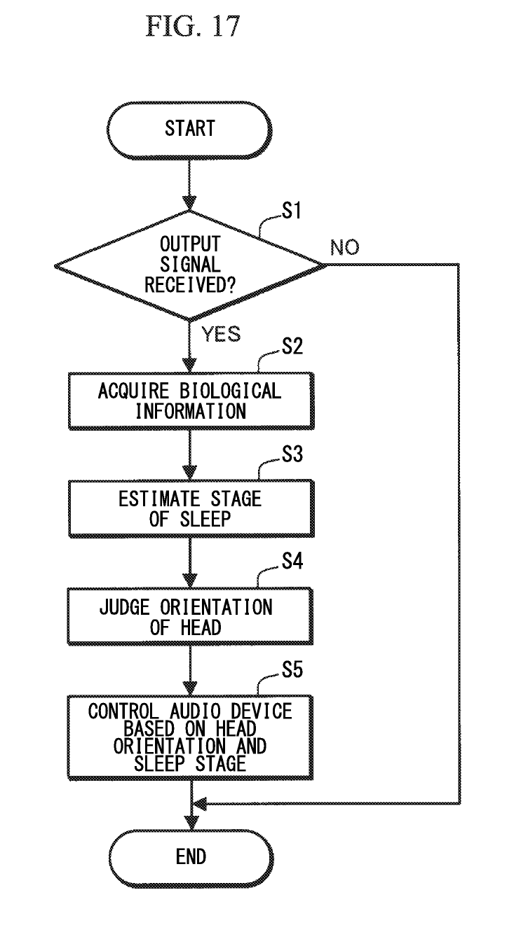

FIG. 17 is a flowchart for describing the operation of the device control apparatus 2100. The device control apparatus 2100 repeats the operation shown in FIG. 17.

When the receiving unit 221R receives the output signal DS-R and the receiving unit 221L receives the output signal DS-L (Step S1), the output signal DS-R is outputted to the biological information acquiring unit 222 and the judging unit 223, and the output signal DS-L is outputted to the biological information acquiring unit 222 and the judging unit 223.

The biological information acquiring unit 222, upon receiving the output signal DS-R and the output signal DS-L, acquires the biological information from the output signal DS-R and the output signal DS-L (Step S2). The biological information acquiring unit 222 outputs the biological information to the estimating unit 2241.

The estimating unit 2241, upon receiving the biological information, estimates the stage of sleep of the user 2E from among three stages on the basis of the biological information (Step S3). The estimating unit 2241 outputs the stage of sleep of the user 2E to the audio device control unit 2242.

Meanwhile, the judging unit 223 judges the head 2H orientation on the basis of the output signal DS-R and the output signal DS-L (Step S4).

In Step S4, the judging unit 223 determines the head 2H orientation corresponding to the state of the output signal DS-R and the output signal DS-L with reference to the head orientation judgment table 212.

For example, when the difference between the level (voltage level) of the output signal DS-R and the level (voltage level) of the output signal DS-L (to be referred to as the “level difference” hereinafter) is within a predetermined value, the judging unit 223 determines the head 2H orientation to be “facing up” (refer to FIG. 9).

When the level of the output signal DS-R decreases and the level of the output signal DS-L increases whereby the level difference exceeds the predetermined value, the judging unit 223 determines the head 2H orientation to be “facing left” (refer to FIG. 11).

When the level of the output signal DS-L decreases and the level of the output signal DS-R increases whereby the level difference exceeds the predetermined value, the judging unit 223 determines the head 2H orientation to be “facing right” (refer to FIG. 10).

The judging unit 223, upon determining the head 2H orientation, outputs the head 2H orientation to the audio device control unit 2242.

The audio device control unit 2242 controls the audio device 2500 on the basis of the head 2H orientation and the stage of sleep of the user 2E (Step S5).

In Step S5, first, the audio device control unit 2242, referring to the device control table 213, sets the loudspeaker that outputs the L sound and the loudspeaker that outputs the R sound.

For example, in Step S5, when the head 2H orientation is facing up, the audio device control unit 2242 outputs to the audio control unit 2501 facing-up setting information indicating the output of the L sound from the loudspeaker 2502d and the output of the R sound from the loudspeaker 2502c.

When the head 2H orientation is facing left, the audio device control unit 2242 outputs to the audio control unit 2501 facing-left setting information indicating the output of the R sound from the loudspeaker 2502a and the output of the L sound from the loudspeaker 2502b.

When the head 2H orientation is facing right, the audio device control unit 2242 outputs to the audio control unit 2501 facing-right setting information indicating the output of the L sound from the loudspeaker 2502a and the output of the R sound from the loudspeaker 2502b.

In Step S5, the audio device control unit 2242 lowers the volume as the stage of sleep becomes deeper.

For example, in the case of the stage of sleep being the first stage, the audio device control unit 2242 outputs to the audio control unit 2501, as the volume, a first volume instruction signal that indicates a first level of the volume.

In the case of the stage of sleep being the second stage, the audio device control unit 2242 outputs to the audio control unit 2501, as the volume, a second volume instruction signal that indicates a second level of the volume.

In the case of the stage of sleep being the third stage, the audio device control unit 2242 outputs to the audio control unit 2501, as the volume, a third volume instruction signal that indicates a third level of the volume.

The first level is higher than the second level, and the second level is higher than the third level.

The audio control unit 2501, upon receiving the facing-up setting information, supplies the L (left) sound signal corresponding to the L sound to the loudspeaker 2502d and supplies the R (right) sound signal corresponding to the R sound to the loudspeaker 2502c. Therefore, the L sound is output from the loudspeaker 2502d and the R sound is output from the loudspeaker 2502c.

When the user 2E is facing up, the loudspeaker 2502c that outputs the R sound is positioned on the right-ear side of the user 2E, and the loudspeaker 2502d that outputs the L sound is positioned on the left-ear side of the user 2E. For this reason, the user 2E can recognize the sound output by the audio device 2500 as stereo sound.

The audio control unit 2501, upon receiving the facing-left setting information, supplies the R sound signal to the loudspeaker 2502a and supplies the L sound signal to the loudspeaker 2502b. Thereby, the R sound is output from the loudspeaker 2502a and the L sound is output from the loudspeaker 2502b.

When the user 2E is facing left, the loudspeaker 2502a that outputs the R sound is positioned on the right-ear side of the user 2E, and the loudspeaker 2502b that outputs the L sound is positioned on the left-ear side of the user 2E. For this reason, the user 2E can recognize the sound output by the audio device 2500 as stereo sound.

The audio control unit 2501, upon receiving the facing-right setting information, supplies the L sound signal to the loudspeaker 2502a and supplies the R sound signal to the loudspeaker 2502b. Thereby, the L sound is output from the loudspeaker 2502a and the R sound is output from the loudspeaker 2502b.

When the user 2E is facing right, the loudspeaker 2502a that outputs the L sound is positioned on the left-ear side of the user 2E, and the loudspeaker 2502b that outputs the R sound is positioned on the right-ear side of the user 2E. For this reason, the user 2E can recognize the sound output by the audio device 2500 as stereo sound.

The audio control unit 2501, upon receiving the first volume instruction signal, sets the volume level of the L sound signal and the R sound signal to the first level. Therefore, when the state of the user 2E is the first stage, the audio control unit 2501 can output stereo sound at the first level volume to the user 2E.

The audio control unit 2501, upon receiving the second volume instruction signal, sets the volume level of the L sound signal and the R sound signal to the second level (second level<first level). Therefore, when the state of the user 2E is the second stage, the audio control unit 2501 can output stereo sound at the second level volume to the user 2E.

The audio control unit 2501, upon receiving the third volume instruction signal, sets the volume level of the L sound signal and the R sound signal to the third level (third level<second level). Therefore, when the state of the user 2E is the third stage, the audio control unit 2501 can output stereo sound at the third level volume to the user 2E.

In this way, as the sleep of the user 2E deepens, the volume of the sound output by the audio device 2500 decreases. For this reason, it is possible to lower the possibility of the user 2E who has started to enter sleep being awoken by the sound from the audio device 2500.

When the receiving unit 221 has not received the output signals DS-R and DS-L in Step S1, (Step S1: NO), the operation shown in FIG. 17 ends.

According to the embodiment B1, the device control unit 224 controls the audio device 2500 in accordance with the head 2H orientation. For that reason, even if the head 2H orientation changes, the audio device 2500 can impart a predetermined effect (in this case, the effect of supplying stereo sound to the user 2E) to the user 2E.

The biological information acquiring unit 222 acquires the biological information of the user 2E on the basis of the output signal DS-R and DS-L.

For this reason, it is possible to reduce the number of signals received by the device control apparatus 100 compared to the case of acquiring the biological information of the user 2E on the basis of a signal that differs from both the output signals DS-R and DS-L.

Also, the pressure sensors 2200R and 2200L that are used for judging the head 2H orientation can also be made to serve as sensors for detecting biological information. For this reason, it becomes possible to achieve simplification of the constitution.

The device control unit 224 controls the audio device 2500 on the basis of the biological information acquired by the biological information acquiring unit 222. Therefore, it is possible to control the audio device 2500 in a manner matched with the state of the user 2E.

The judging unit 223 may judge the head 2H orientation of the user 2E using a head orientation judgment model generated by machine learning.

For example, the judging unit 223 generates a head orientation judging model by performing machine learning using as learning data each of the output signals DS-R and DS-L when the user 2E is facing up, the output signals DS-R and DS-L when the user 2E is facing left, the output signals DS-R and DS-L when the user 2E is facing right, and the output signals DS-R and DS-L when the user 2E is facing down (prone). The head orientation judgment model is a model that expresses the relationship between the combination of the output signals DS-R and DS-L and the head 2H orientation of the user 2E.

In Step S4, the judging unit 223 uses the head orientation judgment model to determine the head 2H orientation of the user 2E in accordance with the combination of the output signals DS-R and DS-L. When the head orientation judgment model is used, the head 2H orientation of the user 2E is judged as either “facing up”, “facing left”, “facing right”, or “facing down”. In this case, it is possible to omit the head orientation judgment table 212.

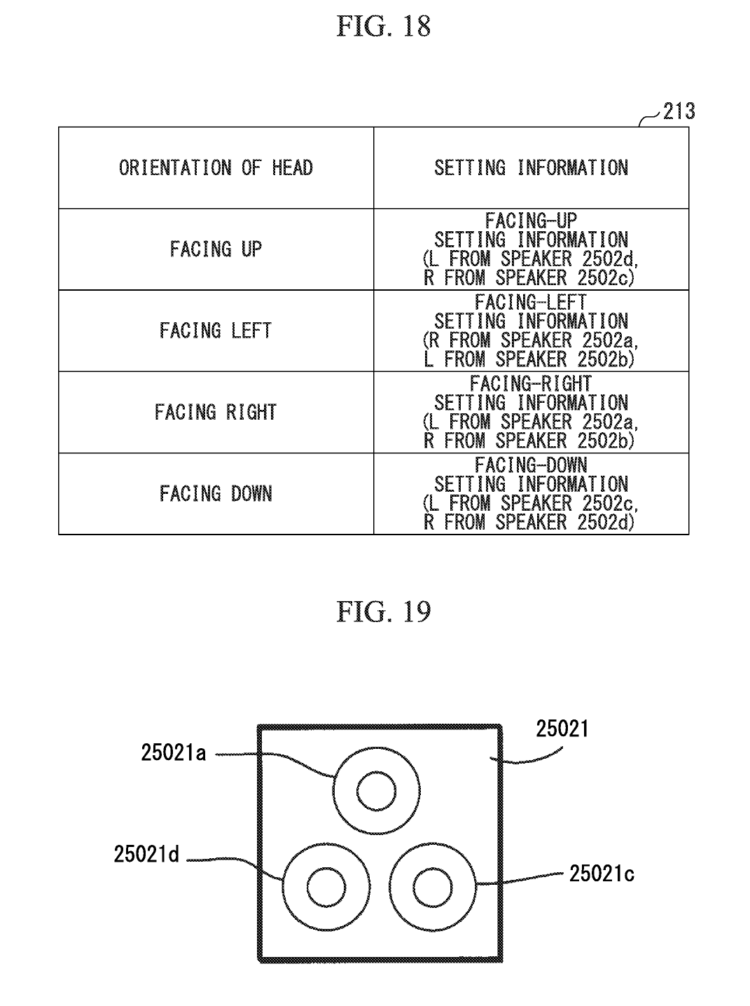

FIG. 18 is a table that shows an example of the device control table 213, which is used when the head 2H orientation has been judged as any one of “facing up”, “facing left”, “facing right”, or “facing down”.

The device control table 213 shown in FIG. 18, in addition to the information stored in the device control table 213 shown in FIG. 16, stores information that shows the correspondence relationship between the orientation “facing down” of the head 2H and the setting information “facing-down setting information”. The facing-down setting information indicates the output of the R sound from the loudspeaker 2502d and the output of the L sound from the loudspeaker 2502c.

In addition, in Step S5, when the head 2H orientation is facing down, the audio device control unit 2242 outputs to the audio control unit 2501 the facing-down setting information. The audio control unit 2501, upon receiving the facing-down setting information, supplies the R sound signal to the loudspeaker 2502d and supplies the L sound signal to the loudspeaker 2502c. Therefore, the R sound is output from the loudspeaker 2502d, and the L sound is output from the loudspeaker 2502c.

When the user 2E is facing down, the loudspeaker 2502d that outputs the R sound is positioned on the right-ear side of the user 2E, and the loudspeaker 2502c that outputs the L sound is positioned on the left-ear side of the user 2E. For this reason, the user 2E can recognize the sound output by the audio device 2500 as stereo sound.

### Embodiment B2

In the embodiment B1, as the loudspeaker unit 2502, a loudspeaker unit having the loudspeakers 2502a and 2502b arranged in a vertical direction and the loudspeakers 2502c and 2502d arranged in a horizontal direction is used. In contrast, in the embodiment B2, a loudspeaker unit having three loudspeakers is used as a loudspeaker unit 25021.

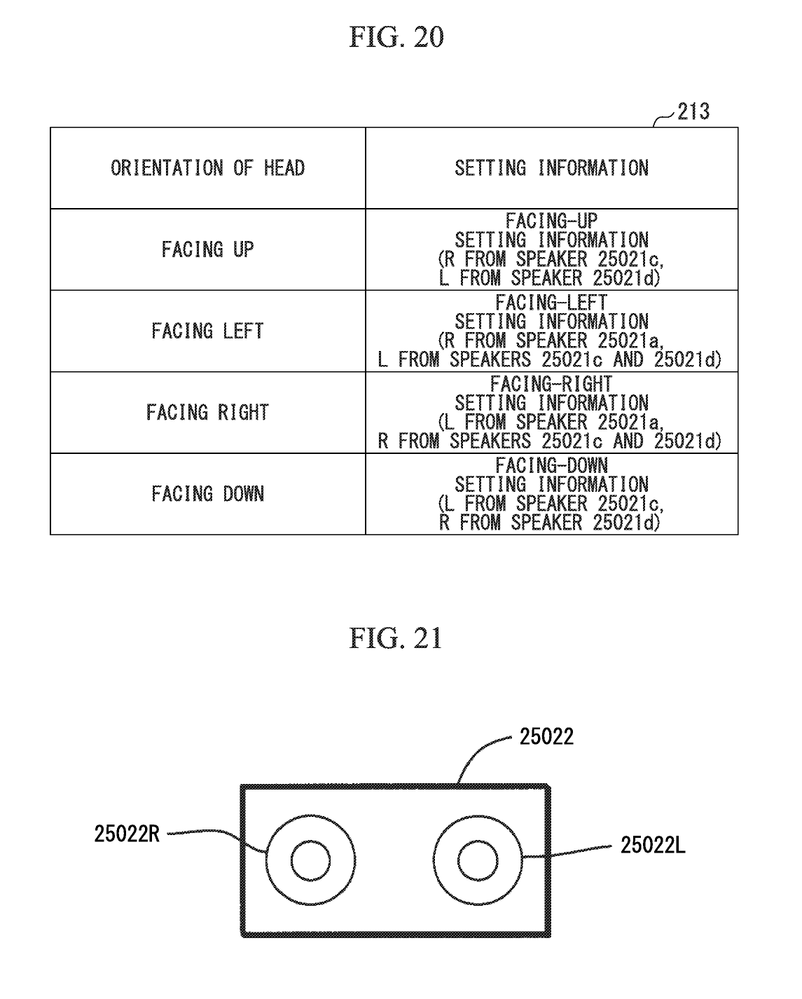

The embodiment B2 differs from the embodiment B1 on the points of a loudspeaker unit including three loudspeakers shown in FIG. 19 being used as the loudspeaker unit 25021, and the device control table shown in FIG. 20 being used as the device control table 213. The embodiment B2 will be described below, focusing on the points of difference with the embodiment B1. The judging unit 223 judges the head 2H orientation as any one of “facing up”, “facing left”, “facing right” and “facing down”.

The loudspeaker unit 25021 shown in FIG. 19 includes loudspeakers 25021a, 25021c and 25021d. The loudspeaker 25021c and the loudspeaker 25021d are arranged in the horizontal direction. The loudspeaker 25021a is disposed at a position shifted upward in the vertical direction from the mid-point between the loudspeaker 25021c and the loudspeaker 25021d.

The device control table 213 shown in FIG. 20 stores, as facing-up setting information, information that indicates setting the loudspeaker 25021c as the loudspeaker that outputs the R sound and setting the loudspeaker 25021d as the loudspeaker that outputs the L sound.

The device control table 213 shown in FIG. 20 stores, as facing-left setting information, information that indicates setting the loudspeaker 25021a as the loudspeaker that outputs the R sound and setting the loudspeakers 25021c and 25021d as loudspeakers that output the L sound.

The device control table 213 shown in FIG. 20 stores, as facing-right setting information, information that indicates setting the loudspeaker 25021a as the loudspeaker that outputs the L sound and setting the loudspeakers 25021c and 25021d as loudspeakers that output the R sound.

The device control table 213 shown in FIG. 20 stores, as facing-down setting information, information that indicates setting the loudspeaker 25021c as the loudspeaker that outputs the L sound and setting the loudspeaker 25021d as the loudspeaker that outputs the R sound.

When the head 2H orientation is facing up, the audio device control unit 2242 outputs facing-up setting information shown in FIG. 20 to the audio control unit 2501. When the head 2H orientation is facing left, the audio device control unit 2242 outputs facing-left setting information shown in FIG. 20 to the audio control unit 2501. When the head 2H orientation is facing right, the audio device control unit 2242 outputs facing-right setting information shown in FIG. 20 to the audio control unit 2501. When the head 2H orientation is facing down, the audio device control unit 2242 outputs facing-down setting information shown in FIG. 20 to the audio control unit 2501.

The audio control unit 2501, upon receiving the facing-up setting information shown in FIG. 20, supplies the R sound signal to the loudspeaker 25021c and supplies the L sound signal to the loudspeaker 25021d. For this reason, the R sound is output from the loudspeaker 25021c, and the L sound is output from the loudspeaker 25021d.

When the user 2E is facing up, the loudspeaker 25021c that outputs the R sound is positioned on the right-ear side of the user 2E, and the loudspeaker 25021d that outputs the L sound is positioned on the left-ear side of the user 2E. For this reason, the user 2E can recognize the sound output by the audio device 2500 as stereo sound.

The audio control unit 2501, upon receiving the facing-left setting information shown in FIG. 20, supplies the R sound signal to the loudspeaker 25021a and supplies the L sound signal to the loudspeakers 25021c and 25021d. For this reason, the R sound is output from the loudspeaker 25021a, and the L sound is output from the loudspeakers 25021c and 25021d.

When the user 2E is facing left, the loudspeaker 25021a that outputs the R sound is positioned on the right-ear side of the user 2E, and the loudspeakers 25021c and 25021d that output the L sound are positioned on the left-ear side of the user 2E. For this reason, the user 2E can recognize the sound output by the audio device 2500 as stereo sound.

The audio control unit 2501, upon receiving the facing-right setting information shown in FIG. 20, supplies the L sound signal to the loudspeaker 25021a and supplies the R sound signal to the loudspeakers 25021c and 25021d. For this reason, the L sound is output from the loudspeaker 25021a, and the R sound is output from the loudspeakers 25021c and 25021d.

When the user 2E is facing right, the loudspeaker 25021a that outputs the L sound is positioned on the left-ear side of the user 2E, and the loudspeakers 25021c and 25021d that outputs the R sound are positioned on the right-ear side of the user 2E. For this reason, the user 2E can recognize the sound output by the audio device 2500 as stereo sound.

The audio control unit 2501, upon receiving the facing-down setting information shown in FIG. 20, supplies the L sound signal to the loudspeaker 25021c and supplies the R sound signal to the loudspeaker 25021d. For this reason, the L sound is output from the loudspeaker 25021c, and the R sound is output from the loudspeaker 25021d.

When the user 2E is facing down, the loudspeaker 25021d that outputs the R sound is positioned on the right-ear side of the user 2E, and the loudspeaker 25021c that outputs the L sound is positioned on the left-ear side of the user 2E. For this reason, the user 2E can recognize the sound output by the audio device 2500 as stereo sound.

Even when three loudspeakers are used as in the embodiment B2, it is possible to have the user 2E recognize the sound output by the audio device 2500 as stereo sound.

The case when the loudspeakers 25021c and 25021d output the L sound, and the 25021a outputs the R sound, and the case when the loudspeakers 25021c and 25021d output the R sound, and the 25021a outputs the L sound, the volume of the sound output by each of the loudspeakers 25021c and 25021d may be made less than the volume of the sound output by the loudspeaker 25021a.

### Embodiment B3

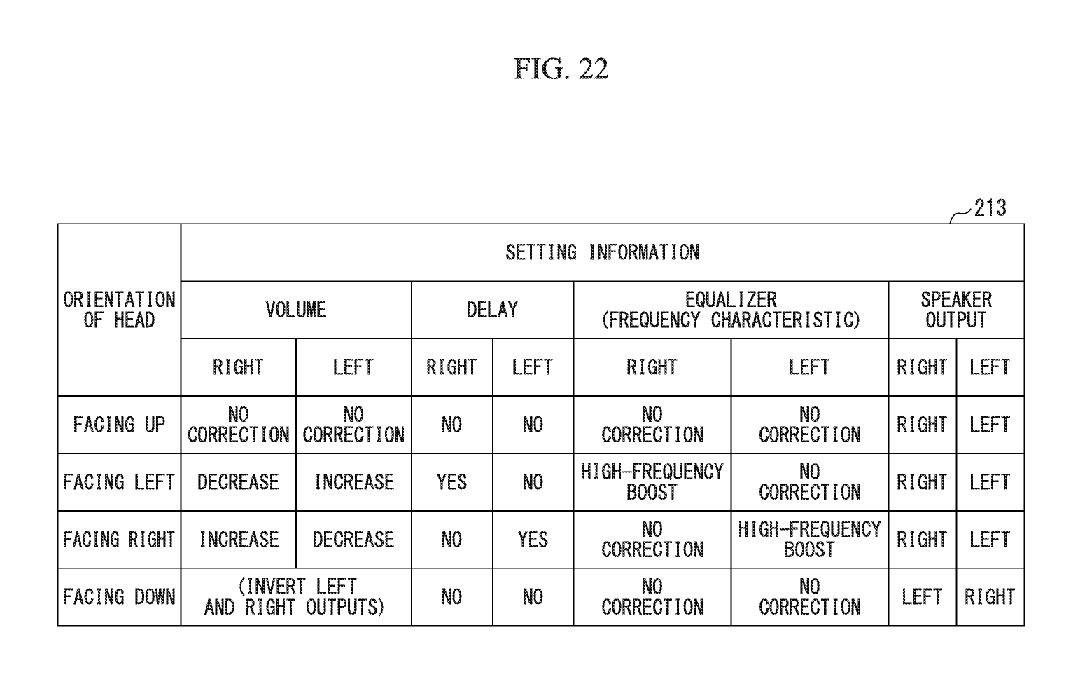

An embodiment B3 differs from the embodiment B1 on the points of a pillow 25022 including two loudspeakers (hereinbelow referred to as a “pillow with loudspeakers”) shown in FIG. 21 used as the loudspeaker unit 2502, the pillow with loudspeakers 25022 used in place of the pillow 252, and a device control table shown in FIG. 22 used as the device control table 213. The embodiment B3 will be described below, focusing on the points of difference with the embodiment B1. The judging unit 223 judges the head 2H orientation as any one of “facing up”, “facing left”, “facing right” and “facing down”.

The pillow with loudspeakers 25022 shown in FIG. 21 includes loudspeakers 25022R and 25022L.

In the state where the user 2E is in a facing-up state on the bed 251 so that the head 2H is in the center of the pillow with loudspeakers 25022, the loudspeaker 25022R is arranged more toward the region that becomes the right-ear side of the user 2E (hereinbelow referred to as the “right-ear side region”) than the center of the pillow 25022.

In the state where the user 2E is in a facing-up state on the bed 251 so that the head 2H is in the center of the pillow with loudspeakers 25022, the loudspeaker 25022L is arranged more toward the region that becomes the left-ear side of the user 2E (hereinbelow referred to as the “left-ear side region”) than the center of the pillow 25022.

As setting information, the device control table 213 shown in FIG. 22 stores, for each head 2H orientation, volume setting information relating to volume, delay setting information relating to delay, frequency characteristic setting information relating to frequency characteristic, and output loudspeaker setting information relating to output loudspeaker.

The setting information shown in FIG. 22 is set on the basis of the relative relation between the distance between the loudspeaker 25022R and the right ear of the user 2E (hereinbelow referred to as the “first distance”) and the distance between the loudspeaker 25022L and the left ear of the user 2E (hereinbelow referred to as the “second distance”). Here, the relative relation between the first distance and the second distance changes in accordance with the head 2H orientation.

For example, when the user 2E is facing up, the difference between the first distance and the second distance is small compared to the case of the user 2E facing right or facing left. For this reason, the difference between the time for sound output from the loudspeaker 25022R to reach the right ear of the user 2E and the time for sound output from the loudspeaker 25022L to reach the left ear of the user 2E is small compared to the case of the user 2E facing right or facing left.

For this reason, when the user is facing up, the volume setting information indicates no correction, the delay setting information indicates no delay, the frequency characteristic setting information indicates no correction, and the output loudspeaker setting information indicates output of the R sound from the loudspeaker 25022R and output of the L sound from the loudspeaker 25022L.

When the user 2E is facing left, the first distance is longer than the second distance. For this reason, in order to overcome the deterioration in the stereo sound caused by the difference between the first distance and the second distance, the volume setting information indicates a decrease in the volume of the R sound by a first predetermined level and an increase in the volume of the L sound by a second predetermined level.

The delay setting information indicates adding a delay of a first time to the R sound and not adding a delay to the L sound.

Since there is a high possibility of the R sound directly reaching the right ear of the user 2E from the pillow with loudspeakers 25022, the frequency characteristic setting information indicates boosting the high-frequency range of the R sound in consideration of the characteristic of the pillow with loudspeakers 25022 and making no correction to the L sound.

The output loudspeaker setting information indicates outputting the R sound from the loudspeaker 25022R and outputting the L sound from the loudspeaker 25022L.

When the user 2E is facing right, the second distance is longer than the first distance. For this reason, the volume setting information, the delay setting information, and the frequency characteristic setting information each indicate setting content opposite to the setting content when the user 2E is facing left. The output loudspeaker setting information is the same as the setting content when the user 2E is facing left.

When the user 2E is facing down, the volume setting information, the delay setting information, and the frequency characteristic setting information each indicate the same setting content as when the user 2E is facing up. The output loudspeaker setting information indicates outputting the R sound from the loudspeaker 25022L and outputting the L sound from the loudspeaker 25022R.

The audio device control unit 2242, in accordance with the head 2H orientation, outputs setting information corresponding to the head 2H orientation (volume setting information, delay setting information, frequency characteristic setting information, and output loudspeaker setting information) among the setting information shown in FIG. 22 to the audio control unit 2501.

The audio control unit 2501, upon receiving the setting information from the audio device control unit 2242, outputs stereo sound in accordance with that setting information.

According to the embodiment B3, it is possible to have the user 2E hear stereo sound by controlling the volume, delay, and frequency characteristic of the stereo sound.

### Modification Examples

The embodiments exemplified above may be modified in various respects. Specific modification examples will be exemplified below. Two or more examples which are arbitrarily selected from modification examples which are exemplified below may be appropriately combined as far as the examples do not conflict with each other.

### Modification Example B1

In the embodiments B1 to B3, the head 2H orientation is judged using a plurality of pressure sensors (pressure sensors 2200R and pressure sensor 2200L). In contrast to this, the head 2H orientation may be judged using one pressure sensor.

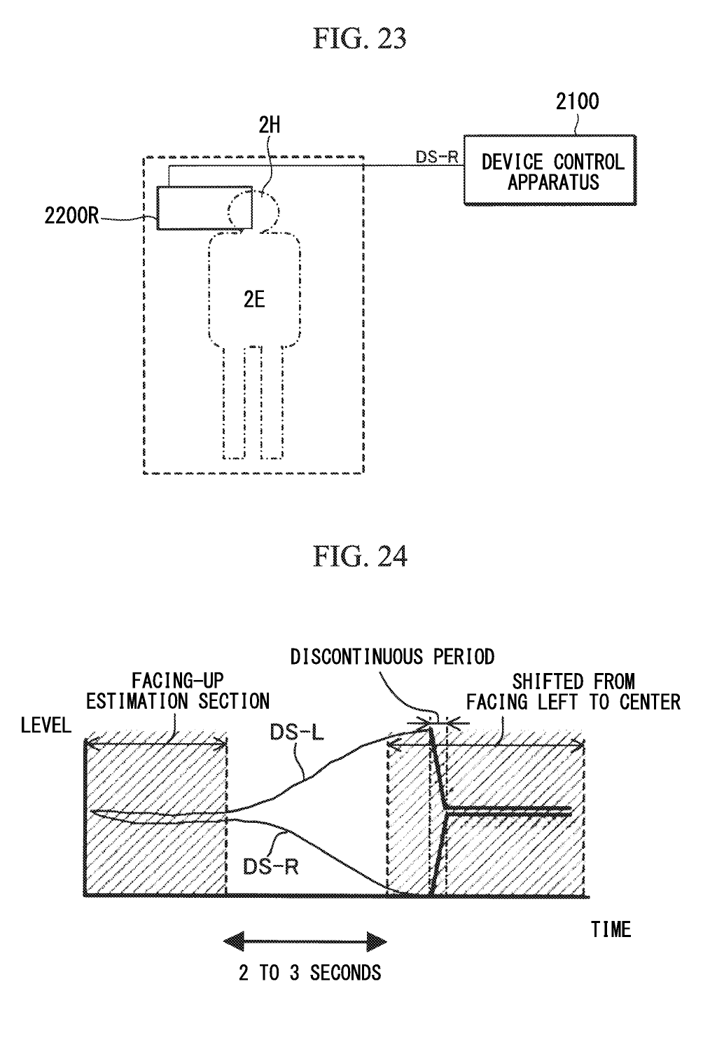

FIG. 23 is a diagram that shows an example of judging the head 2H orientation using the pressure sensor 2200R.

In this example, the judging unit 223 compares the output signal DS-R of the pressure sensor 2200R with a first threshold value and a second threshold value (first threshold value<second threshold value) and judges the head 2H orientation on the basis of the comparison result.

Specifically, when the level of the output signal DS-R is lower than the first threshold value, the judging unit 223 judges that the head 2H is not on the pressure sensor 2200R and therefore that the head 2H is facing left. When the level of the output signal DS-R is equal to or greater than the first threshold value and less than the second threshold value, the judging unit 223 judges that half of the head 2H is on the pressure sensor 2200R and therefore that the head 2H is facing up. When the level of the output signal DS-R is equal to or greater than the second threshold value, the judging unit 223 judges that the entire head 2H is on the pressure sensor 2200R and therefore that the head 2H is facing right.

### Modification Example B2

In the embodiment B3, the device control unit 224 may control one or two of the volume, delay, and frequency characteristic of the stereo sound output from the loudspeaker 25022R and loudspeaker 25022L in accordance with the head 2H orientation to control the sound image of the stereo image output from the loudspeaker 25022R and loudspeaker 25022L.

### Modification Example B3

The control target device is not limited to an audio device and may be appropriately changed. For example, the control target device may be an air conditioner, an electric fan, a lighting device, an elevating bed, or nursing equipment.

For example, when an air conditioner or an electric fan is used as the control target device, as setting information corresponding to the orientation of the head, information that changes the wind direction of the air conditioner or the electric fan to a direction in which the wind from the air conditioner or the electric fan does not directly blow on the face of the user 2E is used. Conversely, as setting information corresponding to the orientation of the head, information that changes the wind direction of the air conditioner or electric fan to a direction in which the wind from the air conditioner or electric fan directly blows on the face of the user 2E may also be used.

### Modification Example B4

The biological information acquiring unit 222 and the estimating unit 2241 may be omitted. In this case, Step S2 and Step S3 in FIG. 17 may be omitted. Therefore, in Step S1, when the receiving unit 221R receives the output signal DS-R and the receiving unit 221L receives the output signal DS-L, Step S4 is executed.

### Modification Example B5

When the user 2E is for example lying down facing left, there is a possibility that the user 2E may shift backward (that is, in the rightward direction when the user 2E is facing up).

FIG. 24 is a diagram that shows an example of the output signal DS-R of the pressure sensor 2200R and the output signal DS-L of the pressure sensor 2200L when the user 2E, while lying down and facing left, has shifted backward (hereinbelow referred to as a “left-facing movement”). As shown in FIG. 24, when a left-facing movement has occurred, there occurs a discontinuous period in the output signal DS-R and the output signal DS-L (that is, a period in which the levels of the output signals do not smoothly change but rather change suddenly). For this reason, when the discontinuous period as shown in FIG. 24 has occurred, the judging unit 223 may judge that the user 2E has performed a left-facing movement and judge that the head 2H orientation is facing left.

A discontinuous period likewise occurs in the output signal DS-R and the output signal DS-L when the user 2E, while lying down facing right, has shifted backward (hereinbelow referred to as a “right-facing movement”). For this reason, when a discontinuous period occurs after the situation in which the relation between the output signal DS-R and the output signal DS-L corresponds to the user 2E facing right, and afterward the difference in the levels of the output signal DS-R and the output signal DS-L is within a predetermined value, the judging unit 223 may judge that the user 2E has performed a right-facing movement and judge that the head 2H orientation is facing right.

### Modification Example B6

All or some of the receiving unit 221, the biological information acquiring unit 222, the judging unit 223, and the device control unit 224 may be realized by dedicated electronic circuits.

### Modification Example B7

In the embodiments B1 to B3 described above, the processing unit 22 of the device control apparatus 2100 controls the audio device 2500, but the embodiments of present invention is not limited thereto. For example, a configuration may be adopted in which for example some functions of the device control apparatus 2100 are provided in an arbitrary server device connected to a communication network (that is, in the cloud), with an information processing device connected with the server device via the same communication network transmitting output signals of the pressure sensors 2200R and 2200L to the server device, and the server device causing the information processing device to control the audio device 2500 via the communication network.

The following modes are ascertained from at least one of the aforementioned embodiments B1 to B3 and the modification examples B1 to B7.

A device control apparatus according to one aspect of the present invention includes: a receiving unit that receives an output signal of a pressure sensor installed in bedding; a judging unit that judges an orientation of a head of a user of the bedding based on the output signal; and a device control unit that controls a control target device in accordance with the orientation of the head.

According to the above device control unit, the control target device can impart a predetermined effect to the user even when the orientation of the user's head changes.

In the above device control apparatus, the control target device may be a sound output apparatus that outputs stereo sound using a plurality of loudspeakers, and the device control unit may control a sound image of the stereo sound that is output from the plurality of loudspeakers in accordance with the head orientation.

According to above device control apparatus, it is possible to have the user hear stereo sound even when the orientation of the user's head changes.

In the above device control apparatus, the device control unit may control at least one of volume, delay, and frequency characteristic of stereo sound output from the plurality of loudspeakers in accordance with the orientation of the head.

According to the above device control apparatus, by controlling at least one of volume, delay, and frequency characteristic of stereo sound output from the plurality of loudspeakers, it is possible to have the user hear stereo sound.

The above device control apparatus may further include an acquiring unit that acquires biological information of the user based on the output signal.

According to the above device control apparatus, it is possible to acquire the biological information of the user from the output signal of a pressure sensor used for judging the orientation of the user's head. Therefore, it is possible to efficiently use the output signal of the pressure sensor compared to the case of acquiring biological information of the user based on a signal that differs from the output signal of the pressure sensor.

The above device control apparatus may further include an acquiring unit that acquires biological information of the user based on the output signal, and the device control unit may further control the sound output apparatus based on the biological information.

According to the above device control apparatus, it is possible to control the sound output apparatus based on the biological information of the user.

A device control method according to one aspect of the present invention includes: receiving an output signal of a pressure sensor installed in bedding; judging an orientation of a head of a user of the bedding based on the output signal; and controlling a control target device in accordance with the orientation of the head.

According to this device control method, the control target device can impart a predetermined effect to the user even when the orientation of the user's head changes.

A device control apparatus according to an aspect of the present invention includes: a receiving unit that receives an output signal of a pressure sensor installed in bedding; a determining unit that determines control information corresponding to the output signal from a plurality of sets of control information for device control; and a device control unit that controls a control target device using the control information corresponding to the output signal.

The above device control apparatus may further includes: an acquiring unit that acquires biological information of a user of the bedding based on the output signal.

In the above device control apparatus, the determining unit may judge an orientation of a head of the user of the bedding based on the output signal. The device control unit may control the control target device in accordance with the orientation of the head.

In the above device control apparatus, the control target device may be a sound output apparatus that outputs stereo sound using a plurality of loudspeakers, and the device control unit may control a sound image of stereo sound output from the plurality of loudspeakers in accordance with the orientation of the head.

In the above device control apparatus, the device control unit may control at least one of volume, delay, and frequency characteristic of stereo sound output from the plurality of loudspeakers in accordance with the orientation of the head.

In the above device control apparatus, the device control unit may control the sound output apparatus further based on the biological information.

A portion of the combination of the judging unit 223 and the device control unit 224 may function as a determining unit that determines setting information (example of control information) corresponding to the output signal received by the receiving unit 221, from a plurality of sets of setting information for device control (example of control information) stored in the device control table (example of control information table) 213. For example, a portion of the combination of the judging unit 223 and the device control unit 224 may function as the above determining unit, by the judging unit 223 judging the orientation of the head 2H of the user 2E based on at least one of the output signal DS-R and the output signal DS-L, and the device control unit 224 determining the setting information corresponding to the judged orientation from a plurality of sets of setting information for device control.

The device control unit 224 may function as a device control unit that controls a control target device using the setting information (example of control information) corresponding to at least one of the output signal DS-R and the output signal DS-L by the device control unit 224 controlling a control target device in accordance with the orientation of the head judged based on at least one of the output signal DS-R and the output signal DS-L.

### Embodiment C1

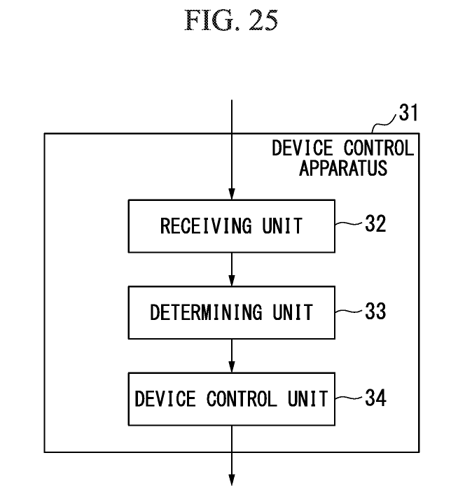

FIG. 25 is a diagram that shows the overall constitution of a device control apparatus 31 according to an embodiment C1 of the present invention. The device control apparatus 31 includes a receiving unit 32, a determining unit 33, and a device control unit 34. The receiving unit 32 receives an output signal of a pressure sensor installed in bedding. The determining unit 33 determines control information corresponding to the output signal from a plurality of sets of control information for device control. The device control unit 34 controls a control target device using the control information corresponding to the output signal.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A device control apparatus comprising:
a receiving unit that receives an output signal of a pressure sensor installed in bedding;
a determining unit that determines control information corresponding to the output signal from a plurality of sets of control information for device control; and
a device control unit that controls a control target device using the control information corresponding to the output signal.

2. The device control apparatus according to claim 1, further comprising:
an acquiring unit that acquires biological information of a user of the bedding based on the output signal.

3. The device control apparatus according to claim 2,
wherein the determining unit comprises:
a tap detecting unit that detects a tap on the bedding based on the output signal; and
a control information determining unit that determines the control information corresponding to the output signal from the plurality of sets of control information, based on the tap.

4. The device control apparatus according to claim 3, wherein the control information determining unit determines the control information corresponding to the output signal from the plurality of sets of control information based on a pattern of the tap.

5. The device control apparatus according to claim 2,
wherein the pressure sensor includes a plurality of first pressure sensors disposed under the bedding so as not to overlap each other, and
the output signal includes a first output signal of each of the plurality of first pressure sensors.

6. The device control apparatus according to claim 2, further comprising:
a sleep judging unit that judges whether the user has gone to sleep based on the biological information,
wherein the determining unit suspends determination of the control information corresponding to the output signal in a case where the sleep judging unit judges that the user has gone to sleep.

7. The device control apparatus according to claim 2,
wherein the determining unit judges an orientation of a head of the user of the bedding based on the output signal, and
the device control unit controls the control target device in accordance with the orientation of the head.

8. The device control apparatus according to claim 7,
wherein the control target device is a sound output apparatus that outputs stereo sound using a plurality of loudspeakers, and
the device control unit controls a sound image of stereo sound output from the plurality of loudspeakers in accordance with the orientation of the head.

9. The device control apparatus according to claim 8, wherein the device control unit controls at least one of volume, delay, and frequency characteristic of stereo sound output from the plurality of loudspeakers in accordance with the orientation of the head.

10. The device control apparatus according to claim 8, wherein the device control unit controls the sound output apparatus further based on the biological information.

11. A device control method comprising:
receiving an output signal of a pressure sensor installed in bedding;
determining control information corresponding to the output signal from a plurality of sets of control information for device control; and
controlling a control target device using the control information corresponding to the output signal.
